(19) United States
(12) Patent Application Publication
(10) Pub. No.: US 2015/0276748 A1
(43) Pub. Date: Oct. 1, 2015

(54) METHODS OF PROGNOSTICATING AND TREATING CANCER

(71) Applicant: CEDARS-SINAI MEDICAL CENTER, Los Angeles, CA (US)

(72) Inventors: Leland W.K. Chung, Beverly Hills, CA (US); Haiyen Zhau, Beverly Hills, CA (US)

(73) Assignee: Cedars-Sinai Medical Center, Los Angeles, CA (US)

(21) Appl. No.: 14/432,453

(22) PCT Filed: Oct. 25, 2013
(86) PCT No.: PCT/US13/66977

§ 371 Date: Mar. 30, 2015

Related U.S. Application Data
(60) Provisional application No. 61/718,626, filed on Oct. 25, 2012.
(60) Provisional application No. 61/867,996, filed on Aug. 20, 2013.

(51) Int. Cl. G01N 33/574; G06F 19/20

(52) U.S. Cl. CPC G01N 33/57488; G06F 19/20; G01N 2333/70575; G01N 2333/71; G01N 2333/912; G01N 2333/475; G01N 2333/4706; G01N 2333/70578; G06F 19/22

(57) ABSTRACT

The present invention relates to the prognosticating survival of subjects with cancer. More specifically, the invention relates to methods and systems to prognosticate cancer patients by assaying RANKL, NRP-1, p-NF-kB, p-c-Met, VEGF and/or RANK expression levels and comparing those levels to reference values to determine the likelihood of survival. The present invention also provides for methods of selecting appropriate therapies for patients based on their prognosis.

### FIELD OF INVENTION

This invention relates to cancer prognosticating, cancer treatment and mechanistic models based on the understanding of mechanisms of cancer progression supported by both clinical and animal models of cancer bone and soft tissue metastases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

After the implementation of prostate-specific antigen (PSA) screening, prostate cancer (PC) diagnosis became much more common. Since one of every 8-10 men diagnosed with PC dies of this disease, it is important to develop effective predictors to select those who need to be treated and avoid unnecessary treatment [1,2]. Over the past decades, many predictive biomarkers, either associated with tissues or in biologic fluids, have been used to try to differentiate indolent from aggressive forms of PC. These markers are categorized broadly as tumor suppressors, oncogenes, transcription factors, and regulators of cellular metabolism, and phenotypes such as cell proliferation, apoptosis, invasion, migration and metastasis [2,3]. However, there remains a need for methods and systems to prognosticate cancer, and particularly PC.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of prognosticating cancer in a subject who is identified as Caucasian-American, comprising: providing a biological sample comprising a tumor cell from the subject; assaying the biological sample for RANKL expression level and/or NRP-1 expression level; comparing the RANKL expression level to a RANKL reference value and/or comparing the NRP-1 expression level to a NRP-1 reference value; and identifying the subject as having a high likelihood of survival if the RANKL expression level is lower than the RANKL reference value and/or the NRP-1 expression level is lower than the NRP-1 reference value, or identifying the subject as having a low likelihood of survival if RANKL expression level is higher than the RANKL reference value and/or the NRP-1 expression level is higher than the NRP-1 reference value.

Various embodiments of the present invention provide for a method of selecting a treatment for and optionally treating a cancer subject who is identified as Caucasian-American, comprising: providing a biological sample comprising a tumor cell from the subject; assaying the biological sample for RANKL expression level and/or NRP-1 expression level; comparing the RANKL expression level to a RANKL reference value and/or comparing the NRP-1 expression level to a NRP-1 reference value; and selecting a first therapy if the subject's RANKL expression level is lower than the RANKL reference value and/or the subject's NRP-1 expression level is lower than the NRP-1 reference value based on the knowledge that subjects have a high likelihood of survival if their RANKL expression level is lower than the RANKL reference value and/or NRP-1 expression level is lower than the NRP-1 reference value, or selecting a second therapy if the subject's RANKL expression level is higher than the RANKL reference value and/or the subject's NRP-1 expression level is higher than the NRP-1 reference value based on the knowledge that subjects have a low likelihood of survival if their RANKL expression level is higher than the RANKL reference value and/or NRP-1 expression level is higher than the NRP-1 reference value.

Various embodiments of the present invention provide for a method of prognosticating cancer in a subject who is identified as African-American, comprising: identifying the subject's Gleason score; providing a biological sample comprising a tumor cell from the subject; assaying the biological sample for nuclear p-c-Met expression level; comparing the nuclear p-c-Met expression level to a nuclear p-c-Met reference value; and identifying the subject as having a high likelihood of survival if the subject's Gleason score is less than 8 and the nuclear p-c-Met expression level is lower than the nuclear p-c-Met reference value, or identifying the subject as having a low likelihood of survival if the subject's Gleason score is >8 and the nuclear p-c-Met expression level is higher than the nuclear p-c-Met reference value.

Various embodiments of the present invention provide for a method of selecting a treatment for and optionally treating a cancer subject who is identified as African-American, comprising: identifying the subject's Gleason score; providing a biological sample comprising a tumor cell from the subject; assaying the biological sample for nuclear p-c-Met expression level; comparing the nuclear p-c-Met expression level to a nuclear p-c-Met reference value; and selecting a first therapy if the subject's Gleason score is less than 8 and the nuclear p-c-Met expression level is lower than the nuclear p-c-Met reference value based on the knowledge that subjects have a high likelihood of survival if their Gleason score is less than 8 and nuclear p-c-Met expression level is lower than the nuclear p-c-Met reference value, or selecting a second therapy if the subject's Gleason score is >8 and the nuclear p-c-Met expression level is higher than the nuclear p-c-Met reference value based on the knowledge that subjects have a low likelihood of survival if their Gleason score is >8 and the nuclear p-c-Met expression level is higher than the nuclear p-c-Met reference value.

Various embodiments of the present invention provide for a method of prognosticating cancer in a subject who is identified as Chinese, comprising: providing a biological sample comprising a tumor cell from the subject; assaying the biological sample for NRP-1 expression level, p-NF-κB p65 expression level, and/or VEGF expression level; comparing the NRP-1 expression level to NRP-1 reference value, p-NF-κB p65 expression level to NF-κB p65 reference value, and/or VEGF expression level to VEGF reference value; and identifying the subject as having a high likelihood of survival if the NRP-1 expression level is lower than the NRP-1 reference value, the p-NF-κB p65 expression level is lower than the p-NF-κB p65 reference value, and/or the VEGF expression level is lower than the VEGF reference value, or identifying the subject as having a low likelihood of survival if the NRP-1 expression level is higher than the NRP-1 reference value, the p-NF-κB p65 expression level is higher than the p-NF-κB p65 reference value, and/or the VEGF expression level is higher than the VEGF reference value.

Various embodiments of the present invention provide for a method of selecting a treatment for and optionally treating a cancer subject who is identified as Chinese, comprising: providing a biological sample comprising a tumor cell from the subject; assaying the biological sample for NRP-1 expression level, p-NF-κB p65 expression level, and/or VEGF expression level; comparing the NRP-1 expression level to NRP-1 reference value, p-NF-κB p65 expression level to NF-κB p65 reference value, and/or VEGF expression level to VEGF reference value; and selecting a first therapy if the subject's NRP-1 expression level is lower than the NRP-1 reference value, p-NF-κB p65 expression level is lower than the p-NF-κB p65 reference value, and/or VEGF expression level is lower than the VEGF reference value based on the knowledge that subjects have a high likelihood of survival if their NRP-1 expression level is lower than the NRP-1 reference value, p-NF-κB p65 expression level is lower than the p-NF-κB p65 reference value, and/or VEGF expression level is lower than the VEGF reference value, or selecting a second therapy if the subject's NRP-1 expression level is higher than the NRP-1 reference value, p-NF-κB p65 expression level is higher than the p-NF-κB p65 reference value, and/or VEGF expression level is higher than the VEGF reference value based on the knowledge that subjects have a low likelihood of survival if their RP-1 expression level is higher than the NRP-1 reference value, p-NF-κB p65 expression level is higher than the p-NF-κB p65 reference value, and/or VEGF expression level is higher than the VEGF reference value.

Various embodiments of the present invention provide for a method of prognosticating cancer in a subject, optionally selecting a treatment for the subject, and optionally administering the treatment to the subject, comprising: providing a biological sample comprising a cancer cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; and identifying the subject as having castration resistant prostate cancer if the p-c-Met expression level is higher than the p-c-Met reference value, the RANKL expression level is higher than the RANKL reference value, and/or the NRP-1 expression level is higher than the NRP-1 reference value.

In various embodiments, the method can further comprise selecting a treatment for the subject, comprising: selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects are unlikely to have castration resistant prostate cancer if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects likely have castration resistant prostate cancer if their p-c-Met expression level is higher than the p-c-Met reference value, RANKL expression level is higher than the RANKL reference value, and/or NRP-1 expression level is higher than the NRP-1 reference value.

Various embodiments of the present invention provide for a method of prognosticating cancer in a subject, optionally selecting a treatment for the subject, and optionally administering the treatment to the subject, comprising: providing a biological sample comprising a cancer cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; and identifying the subject as having a high likelihood of survival if the p-c-Met expression level is lower than the p-c-Met reference value, the RANKL expression level is lower than the RANKL reference value, and/or the NRP-1 expression level is lower than the NRP-1 reference value, or identifying the subject as having low likelihood of survival if the p-c-Met expression level is higher than the p-c-Met reference value, the RANKL expression level is higher than the RANKL reference value, and/or the NRP-1 expression level is higher than the NRP-1 reference value.

In various embodiments, the method can further comprise selecting a treatment for the subject, comprising: selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects have a high likelihood of survival if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects have a low likelihood of survival if their p-c-Met expression level is higher than the p-c-Met reference value, RANKL expression level is higher than the RANKL reference value, and/or NRP-1 expression level is higher than the NRP-1 reference value.

Various embodiments of the present invention provide for a method of prognosticating cancer in a subject, optionally selecting a treatment for the subject, and optionally administering the treatment, comprising: providing a biological sample comprising a cancer-associated stromal cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; and identifying the subject as having a high likelihood of survival if the p-c-Met expression level is lower than the p-c-Met reference value, the RANKL expression level is lower than the RANKL reference value, and/or the NRP-1 expression level is lower than the NRP-1 reference value, or identifying the subject as having a low likelihood of survival if the p-c-Met expression level is higher than the p-c-Met reference value, the RANKL expression level is higher than the RANKL reference value, and/or the NRP-1 expression level is higher than the NRP-1 reference value.

In various embodiments, the method can further comprise selecting a treatment for the subject, comprising: selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects have a high likelihood of survival if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects have a low likelihood of survival if their p-c-Met expression level is higher than the p-c-Met reference value, RANKL expression level is higher than the RANKL reference value, and/or NRP-1 expression level is higher than the NRP-1 reference value.

Various embodiments of the present invention provide for a method of prognosticating cancer in a subject, optionally selecting a treatment for the subject, and optionally administering the treatment to the subject, comprising: providing a biological sample comprising a cancer-associated-stromal cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; and identifying the subject as unlikely to have castration resistant prostate cancer if the p-c-Met expression level is lower than the p-c-Met reference value, the RANKL expression level is lower than the RANKL reference value, and/or the NRP-1 expression level is lower than the NRP-1 reference value, or identifying the subject as likely having castration resistant prostate cancer if the p-c-Met expression level is higher than the p-c-Met reference value, the RANKL expression level is higher than the RANKL reference value, and/or the NRP-1 expression level is higher than the NRP-1 reference value.

In various embodiments, the method can further comprise selecting the treatment for the subject, comprising: selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects are unlikely to have castration resistant prostate cancer if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects likely have castration resistant prostate cancer if their p-c-Met expression level is higher than the p-c-Met reference value, the RANKL expression level is higher than the RANKL reference value, and/or the NRP-1 expression level is higher than the NRP-1 reference value.

Various embodiments of the present invention provide for a method of prognosticating cancer in a subject, optionally selecting a treatment for the subject, and optionally administering the treatment to the subject, comprising: providing a biological sample comprising a non-cancer-associated stromal cell from the subject; assaying the biological sample for p-c-Met expression level, and/or RANK expression level; comparing the p-c-Met expression level to a p-c-Met reference value, RANK expression level to a RANK reference value; and identifying the subject as having a high likelihood of survival if the p-c-Met expression level is lower than the p-c-Met reference value, identifying the subject as having a low likelihood of survival or having castration resistant prostate cancer if the p-c-Met expression level is higher than the p-c-Met reference value, or identifying the subject as unlikely having metastasis if the RANK expression level is lower than the RANK reference value, or identifying the subject as likely having metastasis if the RANK expression level is higher than the RANK reference value.

In various embodiments, the method can further comprise selecting the treatment, comprising: selecting a first therapy if the subject's p-c-Met expression level is lower than the p-c-Met reference value based on the knowledge that subjects have a high likelihood of survival if their p-c-Met expression level is higher than the p-c-Met reference value, or selecting a second therapy if the subject's p-c-Met expression level is higher than the p-c-Met reference value based on the knowledge that subjects have a low likelihood of survival if their p-c-Met expression level is higher than the p-c-Met reference value.

In various embodiments, the method can further comprise selecting the treatment, comprising: selecting a first therapy if the subject's RANK expression level is lower than the RANK reference value based on the knowledge that subjects are unlikely to have metastasis if their RANK expression level is lower than the RANK reference value, or selecting a second therapy if the subject's RANK expression level is higher than the RANK reference value based on the knowledge that subjects likely have metastasis if their RANK expression level is higher than the RANK reference value.

Various embodiments of the present invention provide for a method of prognosticating cancer in a subject, optionally selecting a treatment for the subject and optionally administering the treatment to the subject, comprising: providing a biological sample comprising a morphologically normal gland cell from the subject; assaying the biological sample for NRP-1 expression level; comparing the NRP-1 expression level to a NRP-1 reference value; and identifying the subject as unlikely to have castration resistant prostate cancer if the NRP-1 expression level is lower than the NRP-1 reference value, or identifying the subject as likely having castration resistant prostate cancer if the NRP-1 expression level is higher than the NRP-1 reference value.

In various embodiments, the method can further comprise selecting a treatment for the subject, comprising: selecting a first therapy if the subject's NRP-1 expression level is lower than the NRP-1 reference value based on the knowledge that subjects are unlikely to have castration resistant prostate cancer if their the NRP-1 expression level is lower than the NRP-1 reference value, or selecting a second therapy if the subject's NRP-1 expression level is higher than the NRP-1 reference value based on the knowledge that subjects are likely to have castration resistant prostate cancer if their the NRP-1 expression level is higher than the NRP-1 reference value.

Various embodiments of the present invention provide for a system for prognosticating cancer, comprising: a biological sample obtained from a subject who desires a prognosis regarding a cancer; and one or more assays to determine the level of a biomarker selected from the group consisting of RANKL, NRP-1, p-c-Met, p-NF-κB p65, VEGF, RANK and combinations thereof.

Various embodiments of the present invention provide for a system prognosticating cancer in a subject in need thereof, comprising: a sample analyzer configured to produce a signal for a biomarker selected from the group consisting of RANKL, NRP-1, p-c-Met, p-NF-κB p65, VEGF, RANK and combinations thereof in a biological sample of the subject; and a computer sub-system programmed to calculate, based on the biomarker whether the signal is higher or lower than a reference value.

Various embodiments of the present invention provide for a kit for prognosticating a cancer and/or selecting a treatment for a subject in need thereof, comprising: one or more probes comprising a combination of detectably labeled probes for the detection of RANKL, NRP-1, p-c-Met, p-NF-κB p65, VEGF, and/or RANK.

In various embodiments, the kit can further comprise computer program product embodied in a non-transitory computer readable medium that, when executing on a computer, performs steps comprising: detecting the RANKL, NRP-1, p-c-Met, p-NF-κB p65, VEGF, and/or RANK level in a biological sample from the subject; and comparing the RANKL, NRP-1, p-c-Met, p-NF-κB p65, VEGF, and/or RANK level to a reference value.

In various embodiments, assaying the biological sample can comprise using multispectral spectral imaging analysis. In various embodiments, assaying the biological sample can comprise using multiplexed quantum dot labeling imaging analysis (mQDL).

In various embodiments, the first therapy can be selected from the group consisting of using proactive surveillance network, dietary and life-style interventions, cholesterol lowering drug, and hormonal therapy.

In various embodiments, the second therapy can be selected from the group consisting of surgery, radiation therapy, cytotoxic chemotherapy, platinum-comprising chemotherapies, immunotherapy, bone targeted therapy, androgen receptor inhibitor, radiopharmaceutical, signal transduction inhibitor and combinations thereof.

In various embodiments, the methods can further comprise administering the selected therapy.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts Gleason score box-plots by race.

FIG. 2 depicts log-rank test of overall survival by race including all cases (N=54, number of events=47).

FIG. 3 depicts unmixed NRP-1, p-p65 and VEGF protein expression images from the mQDL of tissues from a Chinese patient who survived for 66 months (long) vs a patient who survived for 2 months (short).

FIG. 4 depicts correlogram with pairwise correlations between Gleason scores and biomarker expression in (cytoplasm+nucleus) for Caucasian-Americans (N=20). The main diagonal has the covariate name. At the horizontal and vertical intersection of each covariate, Pearson correlation coefficient (center), and the associated p-value (top-right corner) are shown.

FIG. 5 depicts correlogram with pairwise correlations between Gleason scores and biomarker expression in (cytoplasm+nucleus) for African-Americans (N=20). The main diagonal has the covariate name. At the horizontal and vertical intersection of each covariate, Pearson correlation coefficient (center), and the associated p-value (top-right corner) are shown.

FIG. 6 depicts correlogram with pairwise correlations between Gleason scores and biomarker expression in (cytoplasm+nucleus) for Chinese (N=14). The main diagonal has the covariate name. At the horizontal and vertical intersection of each covariate, Pearson correlation coefficient (center), and the associated p-value (top-right corner) are shown.

FIG. 7 depicts overall survival by biomarker expression in (cytoplasm+nucleus) for Caucasian-Americans (N=20, number of events=16). Log-rank test p-value is presented. X-axis is survival time in months. Y-axis is the proportion of surviving.

FIG. 8 depicts unmixed mQDL images of NRP-1 and RANKL expression from representative tissues (one long survival, one short survival) from a Caucasian-American patient who survived for 163 months (long) and a patient who survived for 2 months (short).

FIG. 9 depicts unmixed mQDL image of p-c-Met protein expression in an African-American patient who survived for 85 months (long) vs an African-American patient who survived for 12 months (short).

FIG. 10 depicts overall survival with dummy variables for interaction between binary nuclear p-c-Met biomarker and binary Gleason score for African-Americans. (N=20, number of events=18). Log-rank test p-value is presented. ‘Biomarker High’ indicates biomarker values above the median of the (continuous) biomarker. ‘Biomarker Low’ indicates biomarker values below or equal to the median of the (continuous) biomarker.

FIG. 11 depicts overall survival with dummy variable for Gleason>8, nuclear p-c-Met Biomarker High, for African-Americans (N=20, number of events=18). Log-rank test p-value is presented. ‘Biomarker High’ indicates biomarker values above the median of the (continuous) biomarker. ‘Biomarker Low’ indicates biomarker values below or equal to the median of the (continuous) biomarker.

FIG. 12 depicts how much the biomarker intensity of total (cytoplasmic+nuclear expression) RANKL can predict patient survival. The plots were based on Caucasian-American study (# of total patient=20, # of event=16). Four values (Minimum, 25%, Median and 75% Q, of the biomarker among all the 20 patients respectively) were selected to show their corresponding survival rate with time.

FIG. 13 depicts how much the biomarker intensity of total (cytoplasmic+nuclear expression) Neuropilin-1 can predict patient survival. The plots were based on Caucasian-American study (# of total patient=20, # of event=16). Four values (Minimum, 25%, Median and 75% Q, of the biomarker among all the 20 patients respectively) were selected to show their corresponding survival rate with time.

FIG. 14 depicts how much the biomarker intensity of total (cytoplasmic+nuclear expression) p-c-Met can predict patient survival. The plots were based on Caucasian-American study (# of total patient=20, # of event=16). Four values (Minimum, 25%, Median and 75% Q, of the biomarker among all the 20 patients respectively) were selected to show their corresponding survival rate with time.

FIG. 15 depicts the isolation of CTCs for further molecular characterization. Live CTCs from the first sample from patient 44 (Table 9) were isolated onto a microscope slide and subjected to mQDL staining for the status of a panel of proteins documented to relate to PCa progression. A, Spectral images from two representative CTCs are shown on the top two panels (8 images each that represent the expression level of RANKL, pc-Met, HIF-1a, pp65, NRP-1 and VEGF). B, Quantification of spectral image intensities of the six proteins indicated in Panel A from five stained cells from the same patient. The relative level of gene expression was calculated based on the expression of HIF-1α which was assigned as 1.0.

FIG. 16 depicts an extended RANK-mediated cell signaling network linking gene expression and cell behaviors in PCa cells. RNAseq was conducted using prostate cancer cells, with LNCaP background, overexpressing RANKL to compare with cells transduced with a control neo gene. The plot highlighted the interrelationship of differential gene expression between cells with high RANKL-mediated signal network as opposed to the control cells. Genes associated with EMT, sternness, neuroendocrine, osteomimicry and metastasis were revealed in RANKL-mediated signal network, and these genes are known to be associated with the ability of PCa cells to develop aggressive phenotypes. In addition, we observed a number of LncRNAs either up- or down-regulated. In this figure, genes marked in red represent the up-regulated whereas genes marked in blue represent the down-regulated genes.

FIG. 17 shows enhanced RANKL-RANK signaling in castrated or high cholesterol diet-fed mice: more abundant CTCs correlate with more bone and soft tissue metastases.

FIG. 18 shows the metastasis and castration resistance status of the patients from whom the specimens were obtained.

FIG. 19 shows that in cancer-associated stroma, P-c-Met (N+C), RANKL (N+C), NRP1 N+C) expression correlate with overall survival.

FIG. 20 shows that in non-cancer-associated stroma: p-c-Met (N+C) expression correlate with overall survival.

FIG. 21 shows that overall survival of patients correlates with the protein expression of p-c-Met, RANKL, and NRP1 across the ethnicities.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

“Cancer” and “cancerous” refer to or describe the physiological condition in mammals that is typically characterized by unregulated cell growth. Examples of cancer include, but are not limited to, breast cancer, colon cancer, lung cancer, prostate cancer (including but not limited to androgen-dependent prostate cancer, castration resistant prostate cancer, androgen-independent prostate cancer, metastatic prostate cancer), hepatocellular cancer, gastric cancer, pancreatic cancer, cervical cancer, ovarian cancer, liver cancer, bladder cancer, kidney cancer, cancer of the urinary tract, thyroid cancer, renal cancer, carcinoma, melanoma, head and neck cancer, and brain cancer (including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas).

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Therapeutically effective amount” as used herein refers to that amount which is capable of achieving beneficial results in a patient; for example, a patient with cancer. A therapeutically effective amount can be determined on an individual basis and will be based, at least in part, on consideration of the physiological characteristics of the mammal, the type of delivery system or therapeutic technique used and the accumulated time of administration relative to the progression of the disease.

The inventors combined cell culture models with lineage relationship, i.e., which share the same genetic background but differ in their aggressiveness, with animal models that display variability in their intrinsic invasiveness and metastatic potential to develop relevant cell signaling pathways closely mimicking the phenotypes and behaviors of clinical human prostate cancer (PC). The inventors conducted a comparative study using clinical PC tissues associated with known patient survival to test the inventors' belief that the expression of certain cell signaling network biomarkers found in animal models driving PC cells to develop lethal bone and soft tissue metastases can be used as biomarkers to predict the progression and survival of PC patients. The inventors sought a better understanding of potential interracial differences of cell signaling networks. While not wishing to be bound by any particular theory, the inventors believe that different RANK- and c-Met-mediated downstream cell signaling components predict the survival of prostate cancer patients with different racial backgrounds.

The inventors previously reported that lethal PC progression to bone and soft tissue metastases is determined by the osteomimetic property of PC cells [4,5]. The inventors found that soluble factors such as β2-microglobulin (β2-M) and receptor activator of NF-κB ligand (RANKL) can drive PC and other human cancer cells to undergo epithelial-to-mesenchymal transition (EMT) and confer aggressive phenotypes including local invasion and distant metastases [6-12]. Among the cell signaling pathways the inventors have studied the activation of RANKL-RANK signaling was of particular interest because this signaling pathway was activated in both animal models and clinical PC specimens[8,12], and targeting RANKL with an anti-RANKL antibody, denosumab, has been highly effective in blocking the lytic bone lesions associated with men treated with androgen deprivation therapy [13]. RANKL-RANK signaling also was found to be involved in the expansion of the stem cell niche during the development of hormone-sensitive organs [14,15]. The inventors observed in both LNCaP and ARCaP cell and animal models that a “vicious cycle” of RANKL-RANK signaling is responsible for conferring the ability of these cells to grow and metastasize to bone and soft tissues in mice, through the induction of EMT, local invasion and distant metastases [5,8,12]. By genetically inactivating RANK or c-Met receptor, the inventors completely abrogated the ability of these cancer cells to metastasize to bone and soft tissues [16]. The inventors found that through RANKL-RANK signaling a number of transcription factors and target genes were regulated in coordination, resulting in an alteration of the fundamental cellular processes of cancer cells. Notably, the inventors found that RANKL-RANK signaling promotes the expression of RANKL, RANK, and c-Met through increased expression of transcription factors c-Myc/Max [16]. In concert with the activation of RANKL-RANK signaling, the inventors also detected increased expression of VEGF in response to elevated HIF-1α transcription factors [6]. VEGF is a critical pro-angiogenic factor that induces proliferation and migration of endothelial cells within tumor vasculature [17]. Aberrant expression of VEGF and its receptors is associated with poor prognosis manifested by increased tumor vascularity, chemoresistance, local tumor invasion and distant metastases [18]. Elevated HIF-1α binds to the hypoxia-response elements (HREs) and activates VEGF promoter [19]. Neuropilin 1 (NRP-1), a VEGF co-receptor, was originally identified as a receptor for semaphorin 3, mediating neuronal guidance and axonal growth [20], that binds specifically VEGF165 but not VEGF121 on the cell surface of endothelial and tumor cells [20,21]. NRP-1 lacks a typical kinase domain, and primarily functions as a co-receptor to form ligand-specific complexes. Aberrant upregulation of NRP-1 has been observed in high Gleason grade and metastatic PC and other solid tumors [22,23]. The inventors' lab reported that VEGF regulated an anti-apoptotic Mcl-1 gene through NRP1-dependent phosphorylation of c-Met in PC cells and broadened the function of this protein in cell signaling network [6].

Racial and ethnic differences in PC have been widely reported [24,25]. While limited published data suggest potential differences in selective gene expression between aggressive versus indolent PC, data describing interracial comparisons of gene expression between the prostate glands from African-Americans and Caucasian-Americans are sparse. Kwabi-Addo and colleagues [26] reported differences in the specific promoter methylation of genes such as GSTPi, AR, RAR beta2, SPARC, TIMP3, and NKX2-5 in which higher methylation was found in African-Americans than in Caucasian-Americans. Using an immunohistochemical staining approach to profile PC specimens obtained from Caucasian-Americans, African-Americans, Chinese and Japanese, the inventors found remarkable differences between these interracial groups with respect to their staining profiles of tumor suppressors, angiogenic and neuroendocrine factors [27,28]. In the present study, the inventors focus their attention on comparing RANKL-RANK signaling and its downstream effectors among Caucasian-Americans, African-Americans, and Chinese because of the significance of this signaling pathway in conferring PC bone and soft tissue metastases [5,6,8,12]. The inventors analyzed the levels of gene expression at a single cell level in clinical specimens obtained from these interracial groups using an established multiplexed quantum dot labeling (mQDL) technique to sequentially label each of the six signaling intermediates, capture multiple images, unmix and quantify the signals at the sub-cellular level and subject the data to a series of logistic statistical analyses to determine their predictive significance either alone or in combination with the clinical Gleason scores. Results of this study demonstrated that different downstream effectors of the RANKL-RANK signaling pathway can predict PC overall survival in interracial groups with PC.

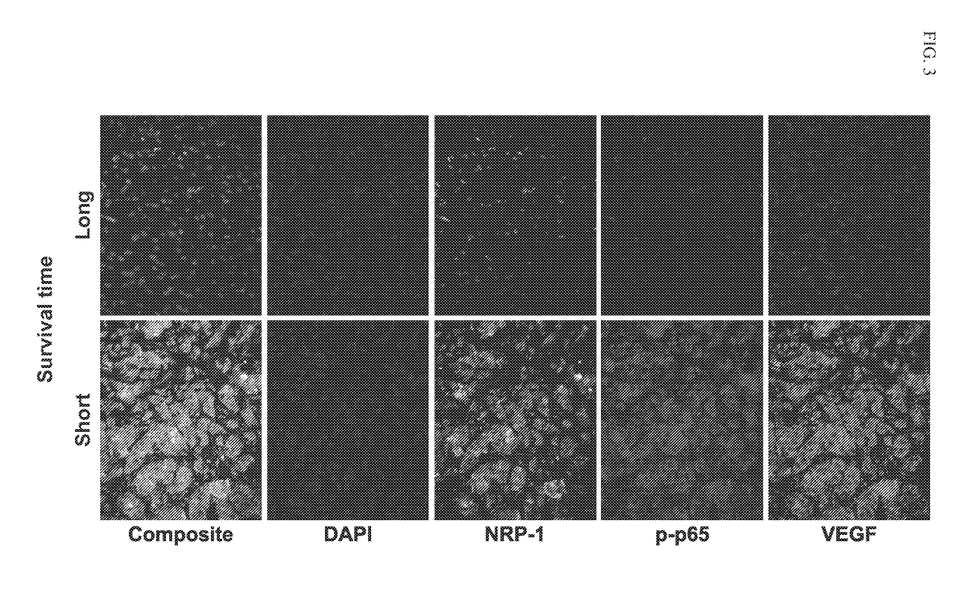

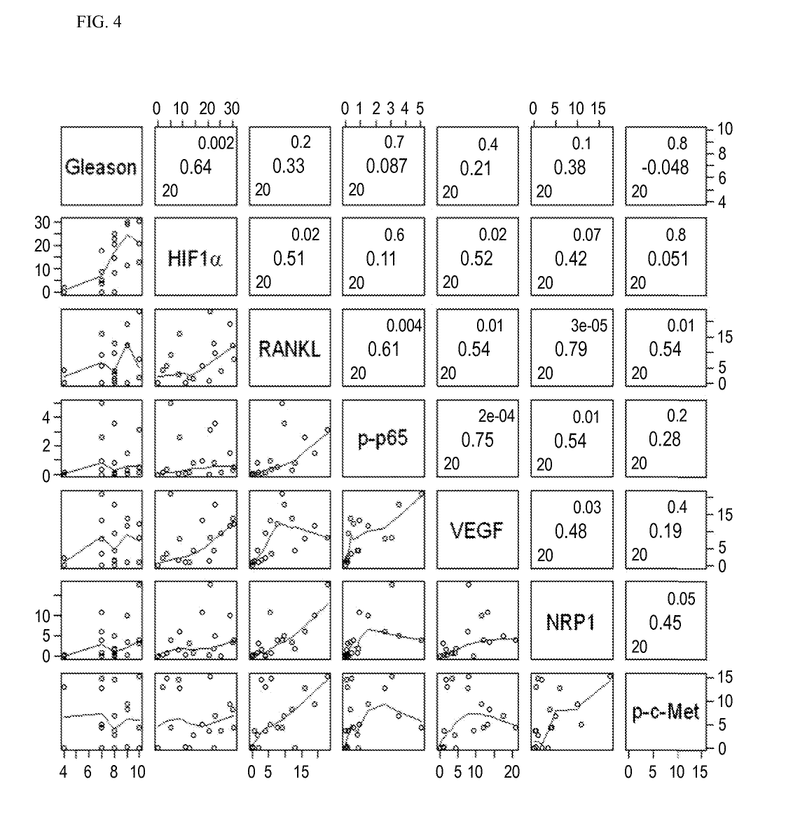

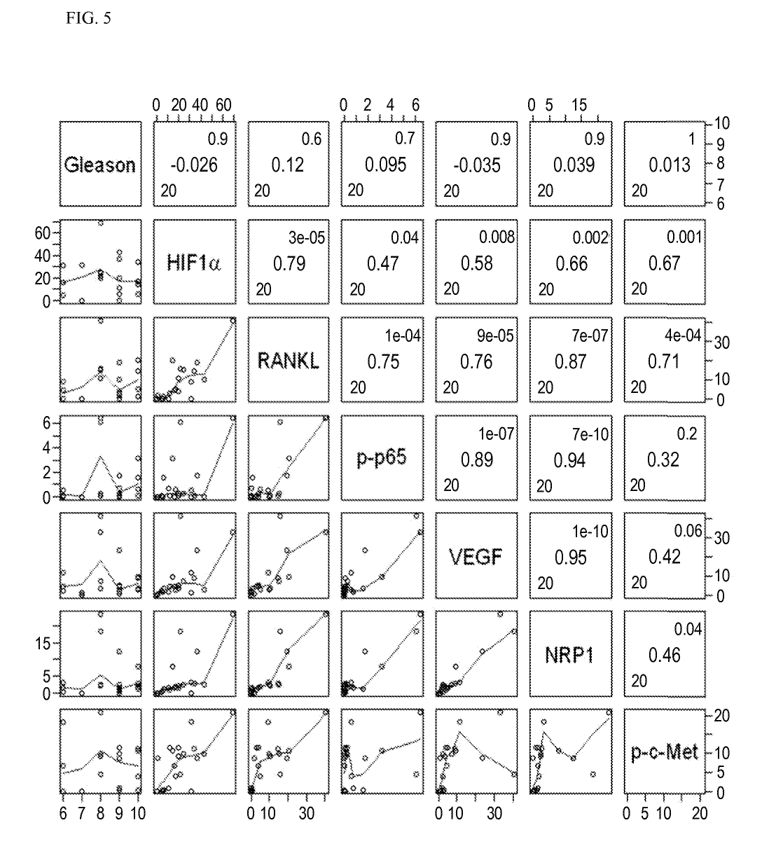

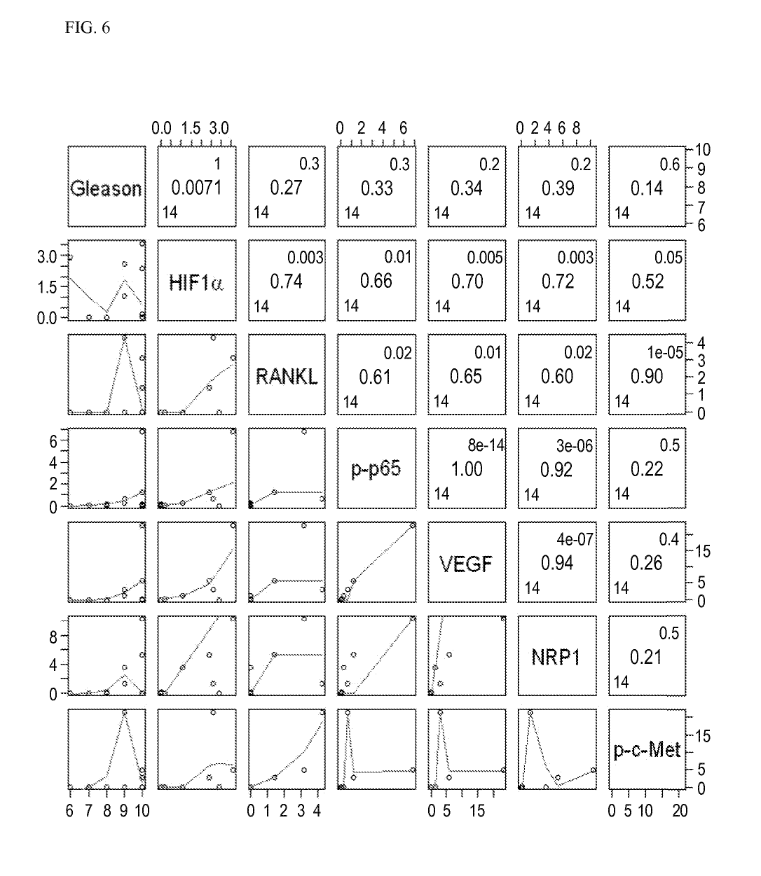

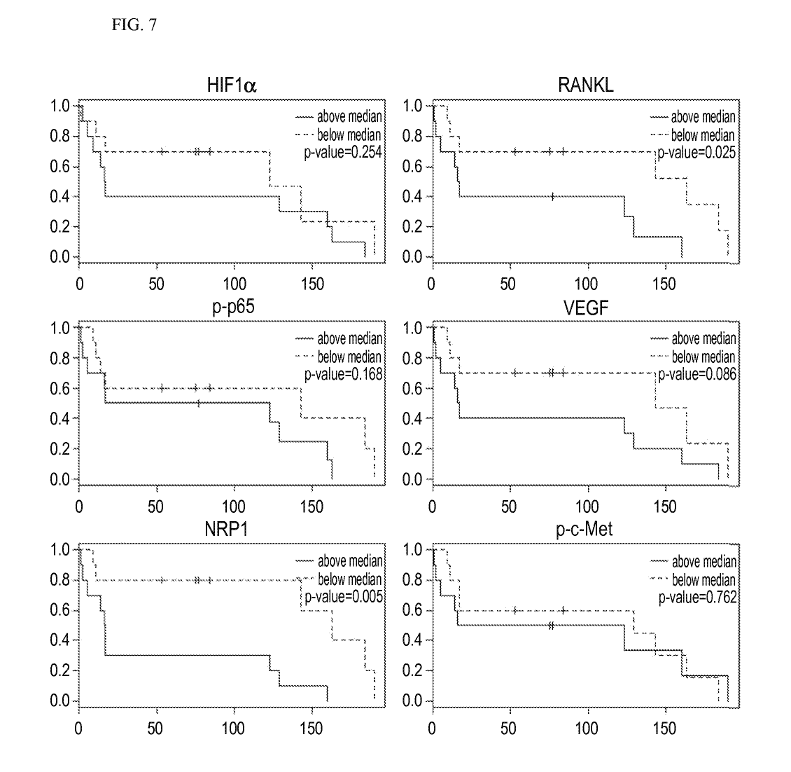

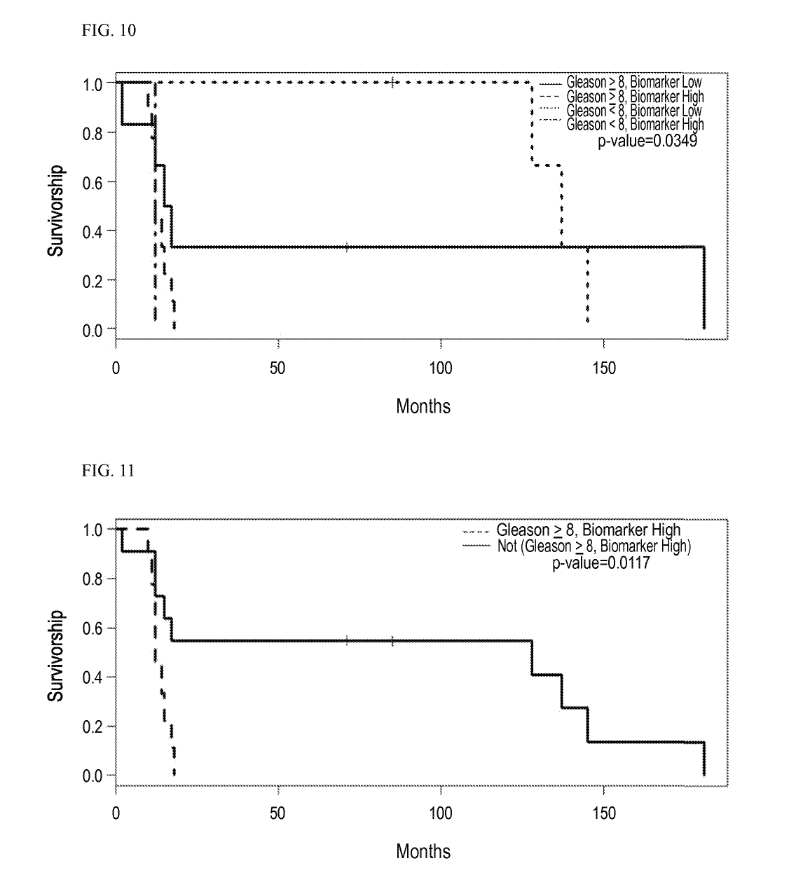

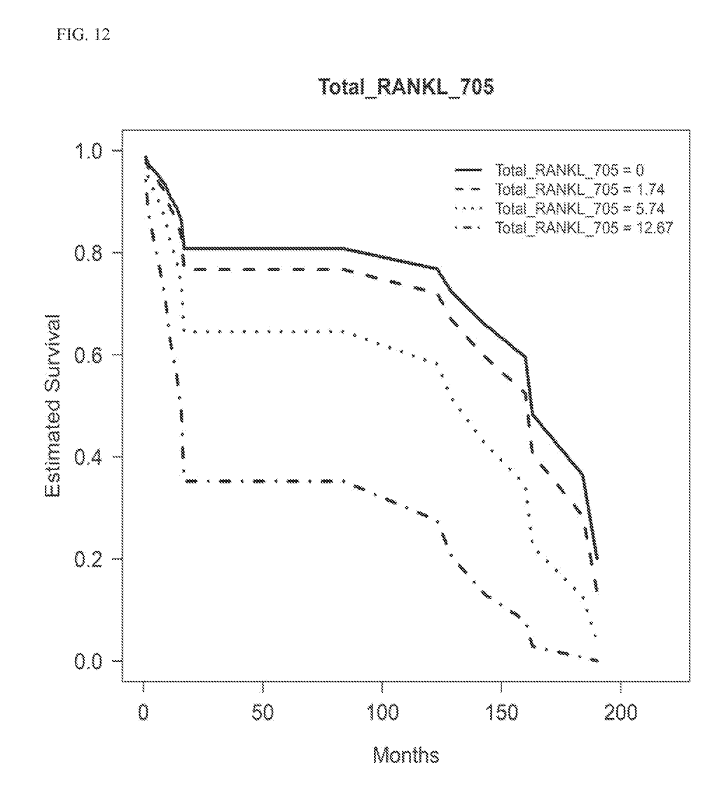

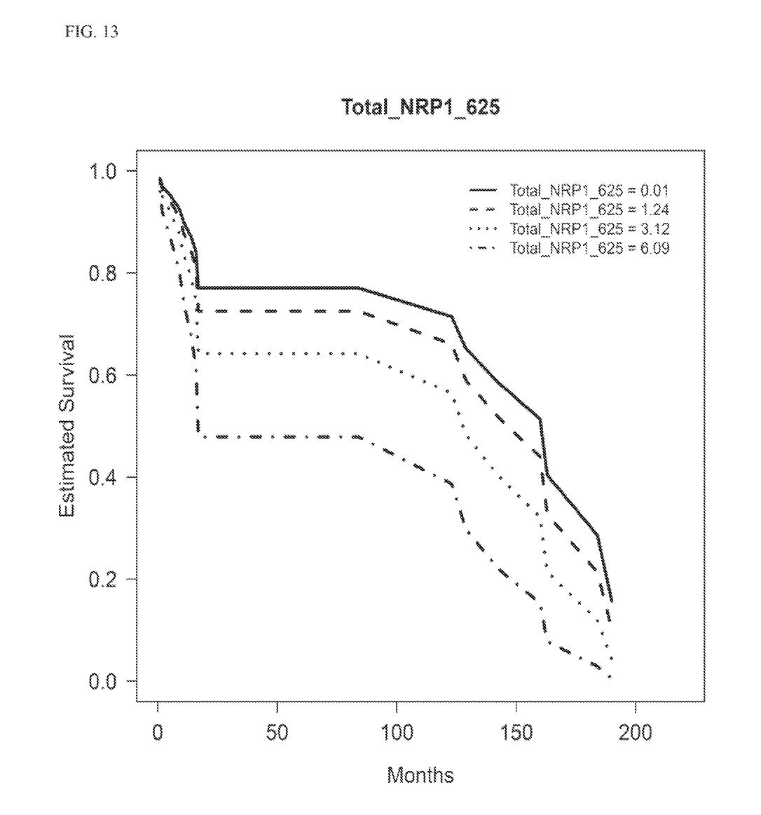

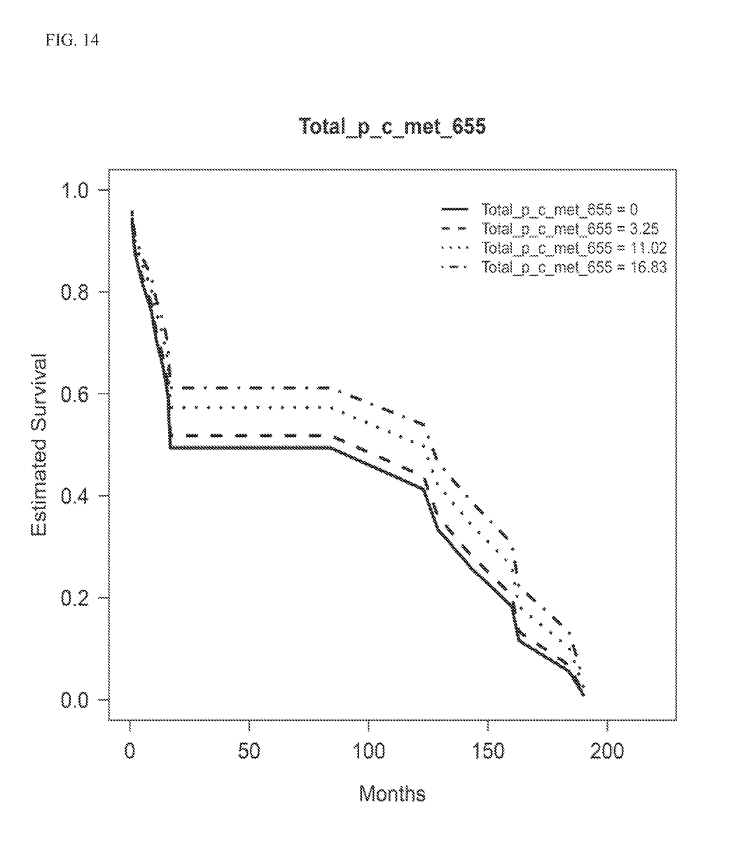

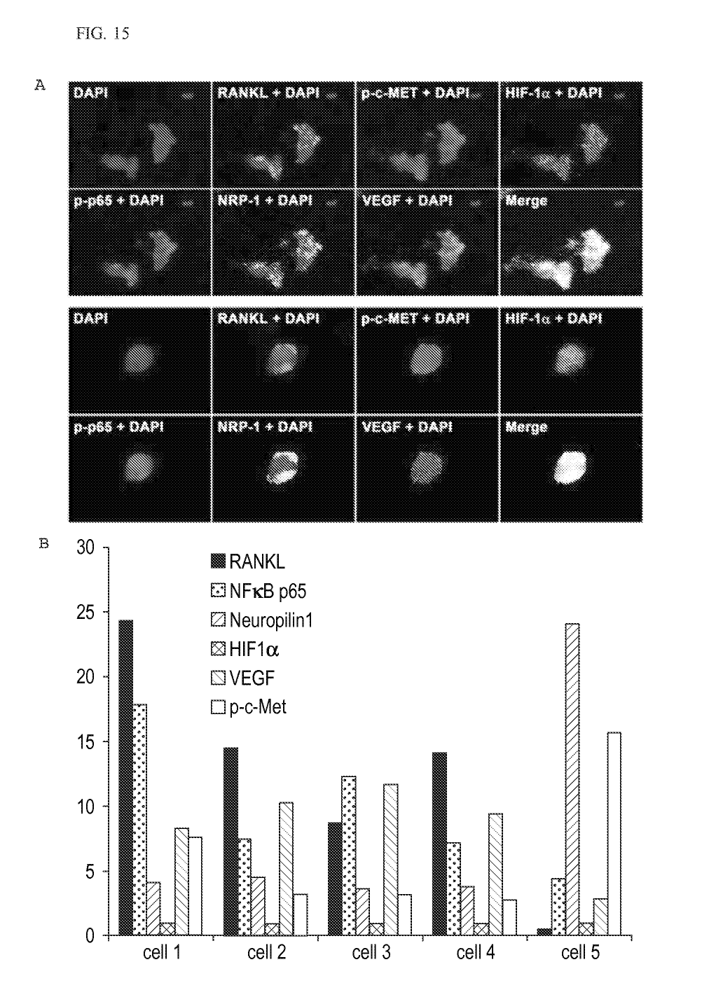

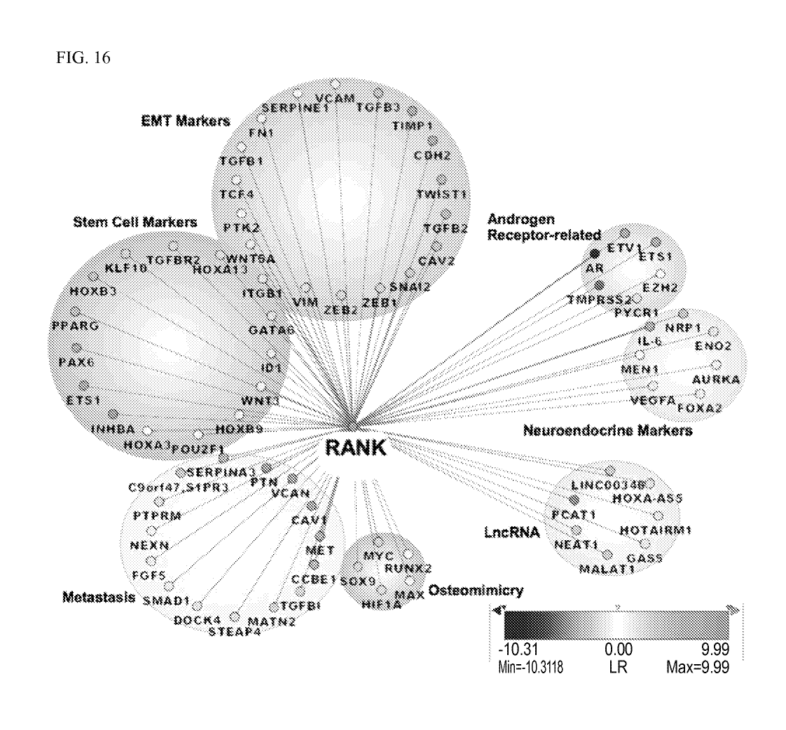

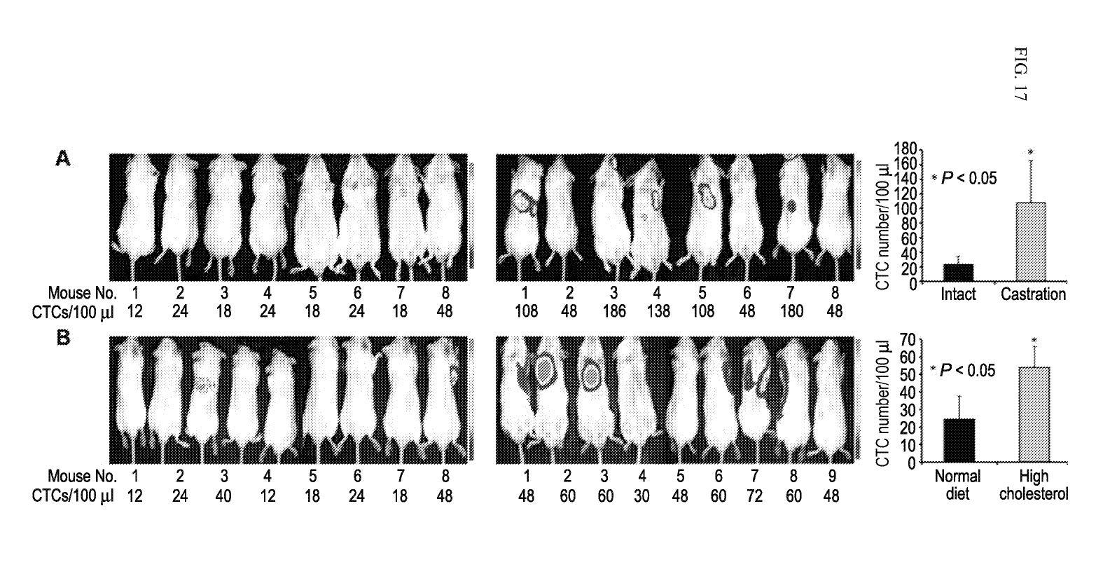

Increasing evidence suggests that improved methods for prognosticating prostate cancer using biomarkers differentially expressed in tissues, cells and body fluids among patients with either indolent or aggressive disease could reduce healthcare costs and patient anxiety and suffering, and improve the overall effectiveness of the treatment plan. While histopathology and immunohistochemistry have provided the “gold standards” for PC diagnosis at the cellular level, the quantitative and prognostic aspects of these techniques have not been critically evaluated. As described herein, the inventors used a multiplexed quantum-dot labeling (mQDL)-based quantitative histopathology approach at a single cell level as reported previously by the inventors' group [6] to assess the expression of cell signaling pathway components downstream from a RANK- and c-Met-mediated signaling network in clinical PC specimens collected from interracial groups, comprised of Caucasian-Americans, African-Americans and Chinese patients, and assessed if these signaling pathway components can predict the survival of PC patients. Activation of RANK- and c-Met-mediated signaling by tumor- and host-derived RANKL has been shown to drive cancer bone and soft tissue metastases in human prostate, breast, lung, kidney and liver cancers. Upon activation of these signaling pathways, the inventors noted increased expression of HIF-1α, VEGF, NRP-1, RANKL, c-Met, and phosphorylated c-Met in cells that conferred resistance to castration and development of a metastatic phenotype in a human PC xenograft model [6]. The inventors found the following interracial differences in the activation of RANK- and c-Met-mediated downstream cell signaling networks in PC cells. 1) RANKL and NRP-1 expression predicts survival of Caucasian-Americans with PC (FIG. 7). 2) In African-Americans, combined Gleason score >8 and nuclear p-c-Met expression predicts survival (FIGS. 10 &11). 3) The inventors found that NRP-1, p-NF-κB p65 and VEGF are predictors for overall survival in Chinese men with PC (Table 3; FIG. 3). 4) Despite differences in the prediction of overall survival of PC patients by different signaling pathway components, all racial groups shared the common downstream signaling components following activation of RANK- and c-Met-mediated signaling. This is revealed by the highly significant pairwise correlation among these signaling components plotted by the Correlogram (FIGS. 4-6) with pair-wise correlations in all three racial groups. Although at the present time there is no scientific explanation for why different signaling components predict survival in three distinct racial groups of PC patients, without being limited by any particular theory, the inventors believe that the regulatory elements, including quantitative aspects of receptors, ligands, and interactions among effector molecules, controlling overall RANK- and c-Met-mediated downstream signaling could be different among interracial groups. These results, however, collectively support interracial differences of RANK- and c-Met-mediated cell signaling network which governs the survival of PC patients.

The present study is the first to use cell-based multispectral quantum dot labeling of rational pathway-associated biomarkers coupled with detailed statistical analyses to test their predicting capability for overall survival of patients with prostate cancer. To reduce the potential variables introduced by tissue specimen processing the inventors chose to use specimens from the same hospital for each racial group. The mQDL and quantification technology demonstrated the predictive utility of RANK- and c-Met-mediated convergent signaling pathways for predicting the overall survival of patients with PC. The inventors' results demonstrated that among the interracial groups, different sets of biomarkers are appropriate for use as predictors for survival. The inventors' findings further support the well documented epidemiological disparities among Caucasian-American, African-American and Chinese patients with PC.

Therefore, embodiments of the present invention are based, at least in part, on these findings described herein.

### Prognosticating Cancer Survival

As discussed above, the prognostication of cancer survival described herein employed biomarkers with the RANK- and cMet-mediated signaling pathway in different interracial groups.

Various embodiments of the present invention provide for a method of prognosticating cancer in a subject who is identified as Caucasian-American, comprising: providing a biological sample comprising a tumor cell from the subject; assaying the biological sample for RANKL expression level and/or NRP-1 expression level; comparing the RANKL expression level to a RANKL reference value and/or comparing the NRP-1 expression level to a NRP-1 reference value; and identifying the subject as having a high likelihood of survival if the RANKL expression level is lower than the RANKL reference value and/or the NRP-1 expression level is lower than the NRP-1 reference value, or identifying the subject as having a low likelihood of survival if RANKL expression level is higher than the RANKL reference value and/or the NRP-1 expression level is higher than the NRP-1 reference value. In particular embodiments the RANKL expression level and/or NRP-1 expression level are RANKL protein expression level and/or NRP-1 protein expression level.

In various embodiments, the RANKL is measured in the nucleus. In other embodiments, the RANKL is measured in the cytoplasm. In other embodiments, the RANKL is measured in the nucleus and the cytoplasm.

In various embodiments, the NRP-1 is measured in the nucleus. In other embodiments, the NRP-1 is measured in the cytoplasm. In other embodiments, the NRP-1 is measured in the nucleus and the cytoplasm.

Various embodiments of the present invention provide for a method of prognosticating cancer in a subject who is identified as Chinese, comprising: providing a biological sample comprising a tumor cell from the subject; assaying the biological sample for NRP-1 expression level, p-NF-κB p65 expression level, and/or VEGF expression level; comparing the NRP-1 expression level to NRP-1 reference value, p-NF-κB p65 expression level to NF-κB p65 reference value, and/or VEGF expression level to VEGF reference value; identifying the subject as having a high likelihood of survival if the NRP-1 expression level is lower than the NRP-1 reference value, the p-NF-κB p65 expression level is lower than the p-NF-κB p65 reference value, and/or the VEGF expression level is lower than the VEGF reference value, or identifying the subject as having a low likelihood of survival if the NRP-1 expression level is higher than the NRP-1 reference value, the p-NF-κB p65 expression level is higher than the p-NF-κB p65 reference value, and/or the VEGF expression level is higher than the VEGF reference value. In particular embodiments, the NRP-1 expression level, p-NF-κB p65 expression level, and/or VEGF expression level are NRP-1 protein expression level, p-NF-κB p65 protein expression level, and/or VEGF protein expression level.

In various embodiments, the NRP-1 is measured in the nucleus. In other embodiments, the NRP-1 is measured in the cytoplasm. In other embodiments, the NRP-1 is measured in the nucleus and the cytoplasm.

In various embodiments, the NF-κB p65 is measured in the nucleus. In other embodiments, the NF-κB p65 is measured in the cytoplasm. In other embodiments, the NF-κB p65 is measured in the he nucleus and the cytoplasm.

In various embodiments, the VEGF is measured in the nucleus. In other embodiments, the VEGF is measured in the cytoplasm. In other embodiments, the VEGF is measured in the nucleus and the cytoplasm.

Various embodiments of the present invention provide for a method of prognosticating cancer in a subject who is identified as African-American, comprising: identifying the subject's Gleason score; providing a biological sample comprising a tumor cell from the subject; assaying the biological sample for nuclear p-c-Met expression level; comparing the nuclear p-c-Met expression level to a nuclear p-c-Met reference value; identifying the subject as having a high likelihood of survival if the subject's Gleason score is less than 8 and the nuclear p-c-Met expression level is lower than the nuclear p-c-Met reference value, or identifying the subject as having a low likelihood of survival if the subject's Gleason score is ≧8 and the nuclear p-c-Met expression level is higher than the nuclear p-c-Met reference value. In various embodiments, the nuclear p-c-Met expression level is nuclear p-c-Met protein expression level.

In other embodiments, the p-c-Met is measured is measured in the cytoplasm. In still other embodiments, the p-c-Met is measured in the nucleus and the cytoplasm.

Various embodiments provide for methods of prognosticating cancer, comprising: providing a biological sample comprising a cancer cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; identifying the subject as likely having castration resistant prostate cancer if the p-c-Met expression level is higher than the p-c-Met reference value, the RANKL expression level is higher than the RANKL reference value, and/or the NRP-1 expression level is higher than the NRP-1 reference value. In various embodiments, the method comprises identifying the subject unlikely to have castration resistant prostate cancer if the p-c-Met expression level is lower than the p-c-Met reference value, the RANKL expression level is lower than the RANKL reference value, and/or the NRP-1 expression level is lower than the NRP-1 reference value

In various embodiments, the p-c-Met is measured in the cytoplasm. In various embodiments, the RANKL is measured in the nucleus, cytoplasm or nucleus and cytoplasm. In various embodiments, the NRP1 is measured nucleus, cytoplasm, or nucleus and cytoplasm. In other embodiments, the p-c-Met, RANKL and NRP1 can be measured in the nucleus, cytoplasm, or nucleus and cytoplasm.

Various embodiments provide for a method of prognosticating cancer in a subject, comprising: providing a biological sample comprising a cancer cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; and identifying the subject as having a high likelihood of survival if the p-c-Met expression level is lower than the p-c-Met reference value, the RANKL expression level is lower than the RANKL reference value, and/or the NRP-1 expression level is lower than the NRP-1 reference value, or identifying the subject as having low likelihood of survival if the p-c-Met expression level is higher than the p-c-Met reference value, the RANKL expression level is higher than the RANKL reference value, and/or the NRP-1 expression level is higher than the NRP-1 reference value.

In various embodiments, the p-c-Met is measured in the cytoplasm. In various embodiments, the RANKL is measured in the nucleus, cytoplasm or nucleus and cytoplasm. In various embodiments, the NRP1 is measured nucleus, cytoplasm, or nucleus and cytoplasm. In other embodiments, the p-c-Met, RANKL and NRP1 can be measured in the nucleus, cytoplasm, or nucleus and cytoplasm.

Various embodiments provide for methods of prognosticating cancer in a subject, comprising: providing a biological sample comprising a cancer-associated stromal cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; identifying the subject as having a high likelihood of survival if the p-c-Met expression level is lower than the p-c-Met reference value, the RANKL expression level is lower than the RANKL reference value, and/or the NRP-1 expression level is lower than the NRP-1 reference value, or identifying the subject as having a low likelihood of survival if the p-c-Met expression level is higher than the p-c-Met reference value, the RANKL expression level is higher than the RANKL reference value, and/or the NRP-1 expression level is higher than the NRP-1 reference value.

In various embodiments, the p-c-Met is measured in the nucleus and cytoplasm. In various embodiments, the RANKL is measured in the nucleus and cytoplasm. In various embodiments, the NRP1 is measured in the nucleus and cytoplasm. In other embodiments, the p-c-Met, RANKL, and NRP1 can be measured in the nucleus, cytoplasm, or nucleus and cytoplasm.

Various embodiments of the present invention provide for methods of prognosticating cancer in a subject, comprising: providing a biological sample comprising a cancer-associated-stromal cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; identifying the subject as unlikely to have castration resistant prostate cancer if the p-c-Met expression level is lower than the p-c-Met reference value, the RANKL expression level is lower than the RANKL reference value, and/or the NRP-1 expression level is lower than the NRP-1 reference value, or identifying the subject as likely having castration resistant prostate cancer if the p-c-Met expression level is higher than the p-c-Met reference value, the RANKL expression level is higher than the RANKL reference value, and/or the NRP-1 expression level is higher than the NRP-1 reference value. In various embodiments, the p-c-Met expression level, RANKL expression level, and/or NRP-1 expression level is p-c-Met protein expression level, RANKL protein expression level, and/or NRP-1 protein expression level

In various embodiments, the p-c-Met is measured in the cytoplasm. In various embodiments, the RANKL is measured in the nucleus, cytoplasm, or nucleus and cytoplasm. In various embodiments the NRP1 is measured in the nucleus, cytoplasm, or nucleus and cytoplasm. In other embodiments, the p-c-Met, RANKL and NRP1 can be measured in the nucleus, cytoplasm, or nucleus and cytoplasm.

Various embodiments provide for methods of prognosticating cancer in a subject, comprising: providing a biological sample comprising a non-cancer-associated stromal cell from the subject; assaying the biological sample for p-c-Met expression level, and/or RANK expression level; comparing the p-c-Met expression level to a p-c-Met reference value, RANK expression level to a RANK reference value; identifying the subject as having a high likelihood of survival if the p-c-Met expression level is lower than the p-c-Met reference value, identifying the subject as having a low likelihood of survival or having castration resistant prostate cancer if the p-c-Met expression level is higher than the p-c-Met reference value, or identifying the subject as unlikely having metastasis if the RANK expression level is lower than the RANK reference value, or identifying the subject as likely having metastasis if the RANK expression level is higher than the RANK reference value. In various embodiments the p-c-Met expression level, and/or RANK expression level is p-c-Met protein expression level, and/or RANK protein expression level

In various embodiments, the p-c-Met is measured in the nucleus and cytoplasm. In various embodiments, the RANK is measured in the nucleus. In other embodiments, the p-c-Met and RANK can be measured in the nucleus, cytoplasm, or nucleus and cytoplasm.

Various embodiments provide for methods of prognosticating cancer in a subject, comprising: providing a biological sample comprising a morphologically normal gland cell from the subject; assaying the biological sample for NRP-1 expression level; comparing the NRP-1 expression level to a NRP-1 reference value; identifying the subject as unlikely to have castration resistant prostate cancer if the NRP-1 expression level is lower than the NRP-1 reference value, or identifying the subject as likely having castration resistant prostate cancer if the NRP-1 expression level is higher than the NRP-1 reference value.

In various embodiments, the NRP-1 is measured in the nucleus. In other embodiments, the NRP-1 can be measured in the cytoplasm, or nucleus and cytoplasm.

Various embodiments of the present invention provide for a system for prognosticating cancer, comprising: a biological sample obtained from a subject who desires a prognosis regarding a cancer; and one or more assays to determine the level of a biomarker selected from the group consisting of RANKL, NRP-1, p-c-Met, p-NF-κB p65, VEGF, RANK and combinations thereof.

Various embodiments of the present invention provide for a system for prognosticating cancer in a subject in need thereof, comprising: a sample analyzer configured to produce a signal for a biomarker selected from the group consisting of RANKL, NRP-1, p-c-Met, p-NF-κB p65, VEGF, RANK and combinations thereof in a biological sample of the subject; and a computer sub-system programmed to calculate, based on the biomarker whether the signal is higher or lower than a reference value. In various embodiments, the system further comprises the biological sample.

Various embodiments of the present invention provide for a computer program product embodied in a non-transitory computer readable medium that, when executing on a computer, performs steps comprising: detecting a biomarker level biomarker selected from the group consisting of RANKL, NRP-1, p-c-Met, p-NF-κB p65, VEGF, RANK and combinations thereof in a biological sample from a subject in need of a prognosis regarding a cancer; and comparing the biomarker level to a reference value.

### Selecting Cancer Treatment and Optionally Administering the Selected Treatment.

Various embodiments the present invention provide for a method of selecting a treatment for and optionally treating a cancer subject who is identified as Caucasian-American, comprising: providing a biological sample comprising a tumor cell from the subject; assaying the biological sample for RANKL expression level and/or NRP-1 expression level; comparing the RANKL expression level to a RANKL reference value and/or comparing the NRP-1 expression level to a NRP-1 reference value; selecting a first therapy if the subject's RANKL expression level is lower than the RANKL reference value and/or the subject's NRP-1 expression level is lower than the NRP-1 reference value based on the knowledge that subjects have a high likelihood of survival if their RANKL expression level is lower than the RANKL reference value and/or NRP-1 expression level is lower than the NRP-1 reference value, or selecting a second therapy if the subject's RANKL expression level is higher than the RANKL reference value and/or the subject's NRP-1 expression level is higher than the NRP-1 reference value based on the knowledge that subjects have a low likelihood of survival if their RANKL expression level is higher than the RANKL reference value and/or NRP-1 expression level is higher than the NRP-1 reference value. In particular embodiments the RANKL expression level and/or NRP-1 expression level are RANKL protein expression level and/or NRP-1 protein expression level.

Various embodiments of the present invention provide for a method of selecting a treatment for and optionally treating a cancer subject who is identified as African-American, comprising: identifying the subject's Gleason score; providing a biological sample comprising a tumor cell from the subject; assaying the biological sample for nuclear p-c-Met expression level; comparing the nuclear p-c-Met expression level to a nuclear p-c-Met reference value; selecting a first therapy if the subject's Gleason score is less than 8 and the nuclear p-c-Met expression level is lower than the nuclear p-c-Met reference value based on the knowledge that subjects have a high likelihood of survival if the subject's Gleason score is less than 8 and the nuclear p-c-Met expression level is lower than the nuclear p-c-Met reference value, or selecting a second therapy if the subject's Gleason score is >8 and the nuclear p-c-Met expression level is higher than the nuclear p-c-Met reference value based on the knowledge that subjects have a low likelihood of survival if the subject's Gleason score is >8 and the nuclear p-c-Met expression level is higher than the nuclear p-c-Met reference value. In particular embodiments, the NRP-1 expression level, p-NF-κB p65 expression level, and/or VEGF expression level are NRP-1 protein expression level, p-NF-κB p65 protein expression level, and/or VEGF protein expression level.

Various embodiments of the present invention provide for a method of selecting a treatment for and optionally treating a cancer subject who is identified as Chinese, comprising: providing a biological sample comprising a tumor cell from the subject; assaying the biological sample for NRP-1 expression level, p-NF-κB p65 expression level, and/or VEGF expression level; comparing the NRP-1 expression level to NRP-1 reference value, p-NF-κB p65 expression level to NF-κB p65 reference value, and/or VEGF expression level to VEGF reference value; selecting a first therapy if the subject's NRP-1 expression level is lower than the NRP-1 reference value, p-NF-κB p65 expression level is lower than the p-NF-κB p65 reference value, and/or VEGF expression level is lower than the VEGF reference value based on the knowledge that subjects have a high likelihood of survival if their NRP-1 expression level is lower than the NRP-1 reference value, p-NF-κB p65 expression level is lower than the p-NF-κB p65 reference value, and/or VEGF expression level is lower than the VEGF reference value, or selecting a second therapy if the subject's NRP-1 expression level is higher than the NRP-1 reference value, p-NF-κB p65 expression level is higher than the p-NF-κB p65 reference value, and/or VEGF expression level is higher than the VEGF reference value based on the knowledge that subjects have a low likelihood of survival if their NRP-1 expression level is higher than the NRP-1 reference value, p-NF-κB p65 expression level is higher than the p-NF-κB p65 reference value, and/or VEGF expression level is higher than the VEGF reference value. In various embodiments, the nuclear p-c-Met expression level is nuclear p-c-Met protein expression level.

In various embodiments, these methods further comprise administering the selected therapy.

Various embodiments provide for methods selecting a treatment for a subject, and optionally administering the treatment to the subject, comprising: providing a biological sample comprising a cancer cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects are unlikely to have castration resistant prostate cancer if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects likely have castration resistant prostate cancer if their p-c-Met expression level is higher than the p-c-Met reference value, RANKL expression level is higher than the RANKL reference value, and/or NRP-1 expression level is higher than the NRP-1 reference value.

In various embodiments, the method further comprises administering the selected therapy.

Various embodiments provide for methods selecting a treatment for the subject, and optionally administering the treatment to the subject, comprising: providing a biological sample comprising a cancer cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; and selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects have a high likelihood of survival if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects have a low likelihood of survival if their p-c-Met expression level is higher than the p-c-Met reference value, RANKL expression level is higher than the RANKL reference value, and/or NRP-1 expression level is higher than the NRP-1 reference value.

In various embodiments, the method further comprises administering the selected therapy.

Various embodiments provide for methods of selecting a treatment for the subject, and optionally administering the treatment, comprising: providing a biological sample comprising a cancer-associated stromal cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects have a high likelihood of survival if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects have a low likelihood of survival if their p-c-Met expression level is higher than the p-c-Met reference value, RANKL expression level is higher than the RANKL reference value, and/or NRP-1 expression level is higher than the NRP-1 reference value.

In various embodiments, the method further comprises administering the selected therapy.

Various embodiments of the present invention provide for methods of selecting a treatment for a subject, and optionally administering the treatment to the subject, comprising: providing a biological sample comprising a cancer-associated-stromal cell from the subject; assaying the biological sample for p-c-Met expression level, RANKL expression level, and/or NRP-1 expression level; comparing the p-c-Met expression level to a p-c-Met reference value, RANKL expression level to a RANKL reference value, and/or NRP-1 expression level to a NRP-1 reference value; selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects are unlikely to have castration resistant prostate cancer if their p-c-Met expression level is lower than the p-c-Met reference value, RANKL expression level is lower than the RANKL reference value, and/or NRP-1 expression level is lower than the NRP-1 reference value, or selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects likely have castration resistant prostate cancer if their p-c-Met expression level is higher than the p-c-Met reference value, the RANKL expression level is higher than the RANKL reference value, and/or the NRP-1 expression level is higher than the NRP-1 reference value.

In various embodiments, the method further comprises administering the selected treatment.

Various embodiments provide for methods of selecting a treatment for a subject, and optionally administering the treatment to the subject, comprising: providing a biological sample comprising a non-cancer-associated stromal cell from the subject; assaying the biological sample for p-c-Met expression level, and/or RANK expression level; comparing the p-c-Met expression level to a p-c-Met reference value, RANK expression level to a RANK reference value; selecting a first therapy if the subject's p-c-Met expression level is lower than the p-c-Met reference value based on the knowledge that subjects have a high likelihood of survival if their p-c-Met expression level is higher than the p-c-Met reference value, or selecting a second therapy if the subject's p-c-Met expression level is higher than the p-c-Met reference value based on the knowledge that subjects have a low likelihood of survival if their p-c-Met expression level is higher than the p-c-Met reference value.

Various embodiments provide for methods of selecting a treatment for a subject, and optionally administering the treatment to the subject, comprising: providing a biological sample comprising a non-cancer-associated stromal cell from the subject; assaying the biological sample for p-c-Met expression level, and/or RANK expression level; comparing the p-c-Met expression level to a p-c-Met reference value, RANK expression level to a RANK reference value; selecting a first therapy if the subject's RANK expression level is lower than the RANK reference value based on the knowledge that subjects are unlikely to have metastasis if their RANK expression level is lower than the RANK reference value, or selecting a second therapy if the subject's RANK expression level is higher than the RANK reference value based on the knowledge that subjects likely have metastasis if their RANK expression level is higher than the RANK reference value.

In various embodiments, the methods further comprise administering the selected therapy.

Various embodiments provide for methods of selecting a treatment for a subject and optionally administering the treatment to the subject, comprising: providing a biological sample comprising a morphologically normal gland cell from the subject; assaying the biological sample for NRP-1 expression level; comparing the NRP-1 expression level to a NRP-1 reference value; selecting a first therapy if the subject's NRP-1 expression level is lower than the NRP-1 reference value based on the knowledge that subjects are unlikely to have castration resistant prostate cancer if their the NRP-1 expression level is lower than the NRP-1 reference value, or selecting a second therapy if the subject's NRP-1 expression level is higher than the NRP-1 reference value based on the knowledge that subjects are likely to have castration resistant prostate cancer if their the NRP-1 expression level is higher than the NRP-1 reference value.

In various embodiments, the method further comprises administering the selected treatment.

In various embodiments, selecting the first therapy can also be based on the knowledge that this class of therapy is appropriate for subjects who have an early onset of disease with high likelihood that this therapy will improve survival and delay disease progression. In various embodiments, selecting the second therapy can be based on the knowledge that this class of therapy is appropriate for subjects who have more advanced disease and this therapy will likely bend the survival curve of the patients.

### Identifying Subjects

In various embodiments of the present invention, the methods are practiced on a subject who is identified as Caucasian-American, African-America, Chinese, Caucasian or

African. Identification of these subjects can be made in a number of ways. For example, identification can be by the subject himself or herself if the subject indicates that he or she is Caucasian-American, African-America, Chinese, Caucasian, or African. Identification can also be made by the practitioner; for example, when a doctor indicates that the subject is Caucasian-American, African-America, Chinese, Caucasian, or African.

### Assaying Biological Samples

One of ordinary skill in the art will readily appreciate methods and systems that can be used to detect the expression level of the biomarkers described herein.

The biological sample can be assayed by various methods. These methods include but are not limited to diaminobenzidine (DAB) immunohistochemical methods, fluorescent immunohistochemical methods, ELISA methods, Western blotting, quantitative reverse transcription polymerase chain reaction (qRT-PCR) of tissue, circulating tumor cells (CTCs), or disseminated tumor cells (DTCs).

These methods and systems also include but are not limited to enzyme-linked immunosorbent assay (ELISA), immunohistochemistry, flow cytometry, fluorescence in situ hybridization (FISH), radioimmuno assays, and affinity purification. Examples of ELISAs include but are not limited to indirect ELISA, sandwich ELISA, competitive ELISA, multiple and portable ELISA.

In various embodiments, assaying the biological sample comprises using multispectral quantitative imaging analysis. In certain embodiments, assaying the biological sample comprises using multiplexed quantum dot labeling. This method is quantitative in comparison to the conventional method for assaying the samples to determine expression levels in tissues, which uses the intensity of IHC staining scored based on a combined intensity and percentage positive scoring cells as previously reported by De Marzo et al. (De Marzo A M, Knudsen B, Chan-Tack K, Epstein J I. E-cadherin expression as a marker of tumor aggressiveness in routinely processed radical prostatectomy specimens. Urology 53(4):707-713, 1999). Recently, however, many other methods have achieved success by using semi-quantitative analyses of gene expression by in situ hybridization, and by the use of apatamer or nanoparticle amplification system. Accordingly, those methods can also be used to detect the expression levels of the biomarkers described herein.

In other embodiments, detecting the expression level of the biomarkers can be done by mass spectrometry (quantitative proteomics). For example, stable (e.g. non-radioactive) heavier isotopes of carbon (¹³C) or nitrogen (¹⁵N) are incorporated into one sample while the other one is labeled with corresponding light isotopes (e.g. ¹²C and ¹⁴N). The two samples are mixed before the analysis. Peptides derived from the different samples can be distinguished due to their mass difference. The ratio of their peak intensities corresponds to the relative abundance ratio of the peptides (and proteins). In various embodiments, isotope labeling can be done by SILAC (stable isotope labeling by amino acids in cell culture), trypsin-catalyzed ¹⁸O labeling, ICAT (isotope coded affinity tagging), iTRAQ (isobaric tags for relative and absolute quantitation).

In other embodiments, a label-free quantitative mass spectrometry can be used to detect the expression level. Spectral counts (or peptide counts) of digested proteins can be used as a way for determining relative protein amounts.

In another embodiment, targeted mass spectrometry can be used. (See e.g., Gillette and Carr, Quantitative analysis of peptides and proteins in biomedicine by targeted mass spectrometry. NAT METHODS. 2013 January; 10(1):28-34).

### Reference Values

In various embodiments of the present invention, the reference value is the average or median RANKL expression of biological samples comprising a tumor cell, a cancer cell, a cancer-associated stromal cell, a non-cancer associated stromal cell, or a morphologically normal gland cell, the biological samples being obtained from cancer subjects. In certain embodiments, the biological sample is the tumor cell, the cancer cell, the cancer-associated stromal cell, the non-cancer associated stromal cell, or the morphologically normal gland cell. In various embodiments, the average or median RANKL expression is the average or median RANKL protein expression.

In various embodiments of the present invention, the reference value is the average or median NRP-1 expression of biological samples comprising a tumor cell, a cancer cell, a cancer-associated stromal cell, a non-cancer associated stromal cell, or a morphologically normal gland cell, the biological samples being obtained from cancer subjects. In certain embodiments, the biological sample is the tumor cell, the cancer cell, the cancer-associated stromal cell, the non-cancer associated stromal cell, or the morphologically normal gland cell. In various embodiments, the average or median NRP-1 expression is the average or median NRP-1 protein expression.

In various embodiments of the present invention, the reference value is the average or median p-c-Met expression from biological samples comprising a tumor cell, a cancer cell, a cancer-associated stromal cell, a non-cancer associated stromal cell, or a morphologically normal gland cell, the biological samples being obtained of cancer subjects. In certain embodiments, the biological sample is the tumor cell, the cancer cell, the cancer-associated stromal cell, the non-cancer associated stromal cell, or the morphologically normal gland cell. In various embodiments, the average or median p-c-Met expression is the average or median p-c-Met protein expression.

In various embodiments of the present invention, the reference value is the average or median p-NF-κB p65 expression of biological samples comprising a tumor cell, a cancer cell, a cancer-associated stromal cell, a non-cancer associated stromal cell, or a morphologically normal gland cell, the biological samples being obtained from cancer subjects. In certain embodiments, the biological sample is the tumor cell, the cancer cell, the cancer-associated stromal cell, the non-cancer associated stromal cell, or the morphologically normal gland cell. In various embodiments, the average or median p-NF-κB p65 expression is the average or median p-NF-κB p65 protein expression.

In various embodiments of the present invention, the reference value is the average or median VEGF expression of biological samples comprising a tumor cell, a cancer cell, a cancer-associated stromal cell, a non-cancer associated stromal cell, or a morphologically normal gland cell, the biological samples being obtained from cancer subjects. In certain embodiments, the biological sample is the tumor cell, the cancer cell, the cancer-associated stromal cell, the non-cancer associated stromal cell, or the morphologically normal gland cell. In various embodiments, the average or median VEGF expression is the average or median VEGF protein expression.

In various embodiments of the present invention, the reference value is the average or median RANK expression of biological samples comprising a tumor cell, a cancer cell, a cancer-associated stromal cell, a non-cancer associated stromal cell, or a morphologically normal gland cell, the biological samples being obtained from cancer subjects. In certain embodiments, the biological sample is the tumor cell, the cancer cell, the cancer-associated stromal cell, the non-cancer associated stromal cell, or the morphologically normal gland cell. In various embodiments, the average or median RANK expression is the average or median RANK protein expression.

The reference value to be used to compare with the expression value of the subject will typically be from the same tissue, cell, and/or location in the cell. For example, if RANKL protein expression level in the nucleus is measured for the subject, it will be compared to RANKL protein expression level in the nucleus of control sample(s). Further, the reference value used can typically be from to control samples having known disease states and survival times.

One of ordinary skill in the art would readily appreciate how to calculate the average or median reference value of the biological samples. The number of subjects from which the reference value is calculated can be, for example, 10, 15, 20, 25, 30, 40, 50, 75, 100, 150, 200, 300, 400, 500, 750, 1000, or more.

In various embodiments, RANKL, NRP-1, p-c-Met, p-NF-κB p65, VEGF, or RANK expression is increased by at least or about 10, 20, 30, 40, 50, 60, 70, 80, or 90% compared to the reference value.

In various embodiments, the RANKL, NRP-1, p-c-Met, p-NF-κB p65, VEGF, or RANK expression is increased by at least or about 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold 2.2-fold 2.3-fold 2.4-fold 2.5-fold, 2.6-fold, 2.7-fold, 2.8-fold, 2.9-fold, or 3-fold, 4-fold, 5-fold, 6-fold, 7-fold, 8-fold, 9-fold or 10-fold compared to the reference value.

In various embodiments, RANKL, NRP-1, p-c-Met, p-NF-κB p65, VEGF, or RANK expression is lower by at least or about 5, 10, 20, 30, 40, 50, 60, 70, 80, 90, 95, 96, 97, 98, or 99% compared to the reference value.

### Biological Sample

The biological sample assayed in the methods and systems of the present invention can be obtained from a subject who desires a prognosis regarding a cancer, a subject who desires the determination of an appropriate therapy to treat the cancer, a subject who desires a determination of whether the cancer is castration resistant prostate cancer, or a subject who desires a determination of whether metastasis has occurred.

Examples of biological samples include but are not limited to body fluids, whole blood, plasma, stool, intestinal fluids or aspirate, and stomach fluids or aspirate, serum, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In particular embodiments of the method, the biological sample may be whole blood, blood plasma, blood serum, bone marrow aspirate, or urine. In certain embodiments, the biological sample is serum.

Additional examples of biological samples include but are not limited to normal tissues, tumor tissues, tumor cells, pathologic samples, bone marrow, bone marrow aspirates, stroma, stromal cells, cancer-associated stroma, cancer-associated stromal cells, non-cancer-associated stroma, non-cancer-associated stromal cells, morphologically normal glands, and morphologically normal gland cells. Still more examples of biological samples include but are not limited disseminated tumor cells (DTCs) (which can be derived from the bone marrow or bone marrow aspirates), and tumor cells in blood circulation (circulating tumor cells (CTCs)).

### Selecting Therapy

Selecting a therapy as used herein, includes but is not limited to selecting, choosing, prescribing, advising, recommending, instructing, or counseling the subject with respect to the treatment.

Various embodiments of the invention involve selecting a first therapy or a second therapy. It is not intended that “first therapy” and “second therapy” refer to trying a certain therapy first and then trying another therapy second. It is used operationally for the convenience of referencing two different classes of therapies and in some cases these two classes of therapies can be used simultaneously in the patients. “First therapy” refers to therapies that are appropriate for subjects who have an early onset of disease with high likelihood that this therapy will improve survival and delay disease progression. “First therapies” are thus appropriate for subjects who have been identified as having a high likelihood of survival by methods of the present invention. “Second therapy” refers to therapies that are appropriate for subject who have more advanced disease and this therapy will likely bend the survival curve of the patients. “Second therapies” are thus appropriate for subjects who have been identified as having a low likelihood of survival by methods of the present invention.

Appropriate “first therapies” include, but are not limited to proactive surveillance network (watchful waiting), dietary and life-style interventions (e.g., low cholesterol food/diet, substitute red meat with seafood, soy based food, green tea, lycopene-rich food, exercise), and hormonal therapy (e.g., finasteride (a 5 alpha reductase inhibitor to block active androgen synthesis)). As discussed herein, a high cholesterol diet increased RANK and RANKL-mediated signaling at the primary and metastatic tumor sites which also reflected in CTCs, a pool of CTCs in exchange with detached cancer cells from either primary or metastatic sites. Thus, a low cholesterol diet can decrease RANKL and RANKL-RANK signaling and be used as a strategy to treat the cancer.

Additionally, cholesterol lowering drugs can be used to treat patients with family history of cancer and high cholesterol. This strategy can be used in combination with dietary control of cholesterol. The cholesterol lowering agents can be selected and administered to lower the cholesterol level of the subject and therefore, lower RANK and RANKL signaling activity. One example of cholesterol lowering drugs is statin drug. Examples of statins include but are not limited to lovastatin (in both immediate release (Mevacor® b.i.d.) and extended release versions (Altoprev®, once daily), pravastatin, atorvastatin, fluvastatin, pitavastatin, rosuvastatin, simvastatin and combination products, including Advicor® (lovastatin/niacin extended release), Simcor® (simvastatin/niacin extended release) and Vytorin® (simvastatin/ezetimibe).

Appropriate “second therapies” include, but are not limited to surgery, radiation therapy, cytotoxic chemotherapy (e.g., Docetaxel, Cabazitaxel, Mitoxantrone, Platinum-comprising chemotherapies (e.g., cisplatin, carboplatin, oxaliplatin, nedaplatin, and iproplatin)), immunotherapy (e.g., Sipuleucel-T, Ipilimumab, ProstVac (PSA-TRICOM vaccine)), bone targeted therapy (e.g., Zoledronic acid, denosumab), Androgen receptor inhibition (e.g., Abiraterone acetate, Enzalutamide (MDV3100), Oteronel (TAK-700), ARN-509, Galeterone (TOK-001)), radiopharmaceuticals (e.g., Alpharadin (Radium-223),

Samarium, Strontium, Lu-177-J591 targeting antibody against a prostate cancer cell surface antigen PSMA, Signal transduction inhibitors (e.g., Dasatinib (a Src-kinase inhibitor), cabozatinib (XL-184), tasquinomod).

The effectiveness of these therapies can be monitored by assessing the inhibition of VEGF, c-Met, RANKL, and hypoxia blockage. Examples of agents that inhibit VEGF, c-Met, RANKL, and hypoxia blockage, include, but are not limited to denosumab, RANK-Fc, OPG-Fc, siRNA, shRNA, XL-184, crizotinib, VEGFR2 kinase inhibitor III (CAS 204005-46-9).

Based on current bone-directed targeting strategies, the following targets downstream from the RANK-mediated signal network can also be selected and administered as a “second therapy”: 1) β2-m. As a pleiotropic signaling molecule for cancer growth and survival, anti-β2-m antibodies or drugs interfering with iron flux can be used in combination with chemotherapy or radiation therapy to enhance the cytotoxicity of antibodies or drugs in tumor cells. 2) c-Met. Using ATP-competitive (PF 02341066, MK-2461) or non-competitive (ARQ-197) c-Met inhibitors, or cabozatinib (XL-184 which is a non-specific receptor tyrosine kinase inhibitor targeting both c-Met and VEGFR2. Additionally, ligand-independent c-Met activation can be blocked by Dasatinib, a Src-kinase inhibitor, that inhibits the ligand-independent activation of c-Me; 3) Inhibition of c-Myc/Max heterodimerization. There are a number of the small molecules modified from the first generation of inhibitor, 10058-F4, and a newer inhibitor of 10074-GS is in the early stages of drug development. 4) Inhibition of EMT by small designed molecules has been shown to have potential for inhibiting epithelium transition to mesenchyme and stem cells. 5) Inhibition of VEGF-neuropilin complex. Small molecules such as EG0229, EG-3287 and VEGF (amino acid-111-165) are under development. In addition to RANK-mediated signal network components, agents interfering with stromal autophagy and miRNA regulators could be used to interfere with RANK-mediated signal networks. These agents can be used in combination with standard hormonal therapy, chemotherapy, immunotherapy and radiation therapy.

In some embodiments wherein the subject has a low likelihood of survival, both a first therapy and a second therapy is selected for and optionally administered to the subject. For example, hormonal therapy and radiation therapy can be selected and administered to the subject.

In various embodiments, the present invention provides pharmaceutical compositions including a pharmaceutically acceptable excipient along with a therapeutically effective amount of an agent of a selected therapy of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch.“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Typical dosages of an effective an agent capable of inhibiting RANK and/or RANKL and/or an agent capable of inhibiting HGF-c-MetNEGFR2/neuropilin-1-mediated signaling including but not limited to downstream activation of neuropilin-1, Src-kinase, Stat3, Mcl-1, and NF-κB of the present invention can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

The present invention is also directed to a kit to prognosticate cancer survival, prognosticate a cancer and/or to select a treatment for a subject. The kit is useful for practicing, for example, the inventive method of identifying a compound that inhibits metastasis or prognosticating a tumor. The kit is an assemblage of materials or components, including at least one of the inventive compositions. Thus, in some embodiments the kit contains a composition including probes and reagents for assaying a biological sample of the present invention, as described above. In some embodiments, the kit contains one or more compositions as discussed above to prognosticate cancer.

The exact nature of the components configured in the inventive kit depends on its intended purpose. For example, some embodiments are configured for the purpose of prognosticating cancer. In one embodiment, the kit is configured particularly for the purpose of prognosticating cancer in mammalian subjects. In another embodiment, the kit is configured particularly for the purpose of prognosticating cancer in human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to prognosticate cancer, or to select a therapy for a cancer subject. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

Various embodiments of the present invention provide for a kit for prognosticating a cancer and/or selecting a treatment for a subject in need thereof, comprising: one or more probes comprising a combination of detectably labeled probes for the detection of RANKL, NRP-1, p-c-Met, p-NF-κB p65, VEGF, and/or RANK. In various embodiments, the kit further comprises the computer program product embodied in a non-transitory computer readable medium that, when executing on a computer, performs steps comprising: detecting the RANKL, NRP-1, p-c-Met, p-NF-κB p65, VEGF, and/or RANK level in a biological sample from a subject in need of a prognosis regarding a cancer; and comparing the RANKL, NRP-1, p-c-Met, p-NF-κB p65, VEGF, and/or RANK level to their respective reference values.

In various embodiments, the kit comprises an assay to detect the levels of the RANKL, NRP-1, p-c-Met, p-NF-κB p65, VEGF, and/or RANK. In various embodiments, the assay comprises a control (e.g., reference value for comparison to the test level).

In various embodiments the kit comprises an assay as discussed herein and instructions to use the assay to prognosticate and/or select a treatment for cancer.

### Non-Human Machines/Computer Implementation Systems and Methods

Various embodiments of the present invention provides for a non-transitory computer readable medium comprising instructions to execute the methods of the present invention, as described herein.

In certain embodiments, the methods of the invention implement a computer program for example, to compare the levels of the biomarkers of the present invention (e.g., RANKL, NRP-1, p-c-Met, p-NF-κB p65, VEGF, or RANK). For example, a non-transitory computer program can be used.

Numerous types of computer systems can be used to implement the analytic methods of this invention according to knowledge possessed by a skilled artisan in the bioinformatics and/or computer arts.

Several software components can be loaded into memory during operation of such a computer system. The software components can comprise both software components that are standard in the art and components that are special to the present invention. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.). In certain embodiments, the computer comprises a database for storage of levels biomarkers of the present invention (e.g., RANKL, NRP-1, p-c-Met, p-NF-κB p65, VEGF, or RANK). Such stored profiles can be accessed and used to compare levels of biomarkers of the present invention (e.g., RANKL, NRP-1, p-c-Met, p-NF-κB p65, VEGF, or RANK) in the sample to known control/reference values.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Once a laboratory technician or laboratory professional or group of laboratory technicians or laboratory professionals determines the level of biomarkers of the present invention (e.g., RANKL, NRP-1, p-c-Met, p-NF-κB p65, VEGF, or RANK), the same or a different laboratory technician or laboratory professional (or group) can analyze one or more assays to determine whether the level of biomarkers of the present invention (e.g., RANKL, NRP-1, p-c-Met, p-NF-κB p65, VEGF, or RANK) differs from the reference value or reference range, and then determine that the subject's prognosis or disease state if the biomarker(s) of the present invention (e.g., RANKL, NRP-1, p-c-Met, p-NF-κB p65, VEGF, or RANK) do differ.

In various embodiments, provided herein is a non-transitory computer readable storage medium comprising: a storing data module containing data from a sample comprising a level of a biomarker of the present invention (e.g., RANKL, NRP-1, p-c-Met, p-NF-κB p65, VEGF, or RANK); a detection module to detect the level of a biomarker of the present invention (e.g., RANKL, NRP-1, p-c-Met, p-NF-κB p65, VEGF, or RANK); a comparison module that compares the data stored on the storing data module with a reference data and/or control data, and to provide a comparison content, and an output module displaying the comparison content for the user, wherein the prognosis or disease state of the is displayed when the level of biomarkers of the present invention (e.g., RANKL, NRP-1, p-c-Met, p-NF-κB p65, VEGF, or RANK) differs from the reference value. In various embodiments, the reference value is a reference range.

In various embodiments, the control data comprises data from patients who do have cancer. In other embodiments, the control data comprises data from patients who do not have cancer.

Embodiments of the invention can be described through functional modules, which are defined by computer executable instructions recorded on a non-transitory computer readable media and which cause a computer to perform method steps when executed. The modules are segregated by function, for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The non-transitory computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can be accessed by a computer including and any suitable combination of the foregoing.

Computer-readable data embodied on one or more non-transitory computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine 2^{nd} ed. (CRC Press, London, 2005) and Ouelette and Bzevanis Bioinformatics: A Practical Guide for Analysis of Gene and Proteins (Wiley & Sons, Inc., 3^{rd} ed., 2004).

The functional modules of certain embodiments of the invention, include for example, a measuring module, a storage module, a comparison module, and an output module. The functional modules can be executed on one, or multiple, computers, or by using one, or multiple, computer networks. The measuring module has computer executable instructions to provide, e.g., expression information in computer readable form.

The measuring module can comprise any system for detecting the levels of biomarkers of the present invention (e.g., RANKL, NRP-1, p-c-Met, p-NF-κB p65, VEGF, or RANK).

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, Blu-ray disc electronic storage media such as RAM, ROM, EPROM, EEPROM and the like, general hard disks and hybrids of these categories such as magnetic/optical storage media. The storage module is adapted or configured for having recorded thereon information on the level of biomarkers of the present invention (e.g., RANKL, NRP-1, p-c-Met, p-NF-κB p65, VEGF, or RANK). Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising information on the levels of the biomarkers of the present invention (e.g., RANKL, NRP-1, p-c-Met, p-NF-κB p65, VEGF, or RANK).

In one embodiment the reference data stored in the storage module to be read by the comparison module is, e.g., data from patients who have cancer and have certain prognosis or certain disease states.

The “comparison module” can use a variety of available software programs and formats for the comparison operative to compare binding data determined in the measuring module to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related, for example, levels of the biomarkers of the present invention (e.g., RANKL, NRP-1, p-c-Met, p-NF-κB p65, VEGF, or RANK).

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using an output module.

The content based on the comparison result, may be levels of the biomarkers of the present invention (e.g., RANKL, NRP-1, p-c-Met, p-NF-κB p65, VEGF, or RANK) compared to reference value(s).

In various embodiments of the invention, the content based on the comparison result is displayed on a computer monitor. In various embodiments of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### PC Tissue Specimens

A total of 54 surgically removed, formalin-fixed and paraffin-embedded (FFPE) primary prostate cancer specimens were obtained from patients from the Department of Pathology, the University of Virginia, Charlottesville, Va., and the Department of Pathology, Jilin University, Changchun, China, with documented cancer-caused death or survival information. Use of clinical specimens was approved by the Institutional Research Board (IRB) of the respective institutions. Of the 54 specimens, 20 each were from Caucasian- and African-Americans and 14 from Chinese men. The number of patients, events, and mean (ranges) survival in months are: Caucasian-Americans—20, 16, and 74.6 (range 1-190); African-Americans—20, 18, and 46.3 (range 2-181); Chinese—14, 13 31.9 (range 1-107). The surgical procedures from which the tissue specimens were obtained were: Caucasian-Americans: 15 cases from transurethral resection of the prostate (TURP), 4 cases from radical prostatectomy (RP) and 1 case from needle biopsy (NBx); African-Americans: 18 cases from TURP and 2 cases from RP; Chinese: 1 case from TURP, 6 cases from suprapubic prostatectomy and 7 cases from NBx. Efforts were made to ensure the consistency of Gleason grading; the histopathologic pattern of the specimens from the U.S. and China were scored by pathologists Dr. L. S. Zhao and Dr. Hua Yang from Jilin University during their visits at UTMDACC in Houston, Tex. and the University of Virginia, respectively, and confirmed by Dr. Henry F. Frierson, a genitourinary pathologist from the University of Virginia.

Immunoassay Reagents

The primary antibodies (Abs) and their sources were: mouse monoclonal Abs against HIF-1α (NB100-105) and RANKL (12A668) from Novus Biologicals (St. Charles, Mo.); rabbit polyclonal Abs to p-NFκB p65 or p-p65 (Ser 536), VEGF (A-20), and neuropilin-1 or NRP-1 (H286) from Santa Cruz Biotechnology, Inc. (Santa Cruz, Calif.); and rabbit polyclonal Ab to p-c-Met (pYpYpY1230/1234/1235) from Invitrogen (Carlsbad, Calif.). Secondary Abs used in the study were prepared in a cocktail of biotinylated Abs to mouse, rabbit, and goat IgG from Vector Laboratories Inc. (Burlingame, Calif.). Phosphate-buffered saline (PBS) and streptavidin-conjugated quantum dots (QD) at 565-, 585-, 605-, 625-, 655- and 705 nm wavelengths as 1 μM stock solution were from Invitrogen.

Multiplexed QD Labeling (mQDL)

The inventors developed a mQDL protocol using streptavidin-coated QDs conjugated to biotinylated secondary Ab [6]. The experimental labeling protocol involved conjugating the primary Ab to a biotinylated secondary Ab, which in turn reacts with streptavidin-conjugated QD at a specified wavelength. This labeling procedure was repeated for multiple primary Abs against different biomarker antigens after optimization. The QD-labeled images were examined and captured under a Nuance multispectral camera and the cellular segmentation and quantification were performed by inForm software (Perkin Elmer; Waltham, Mass.). The multispectral QD image cube was further unmixed to its component images with distinct peak QD wavelengths. After removing the autofluorescence, the individual QD-labeled proteins can be detected.

The immunoreaction sequences and the dilutions of primary Ab and its pairing streptavidin-conjugated QD were: 1) anti-HIF-1α Ab (1:40)) and streptavidin-QD565 (1:100); 2) anti-p-NFκB p65 Ab (1:100) and streptavidin-QD585 (1:100); 3) anti-VEGF Ab (1:40) and streptavidin-QD605 (1:100); 4) anti-neuropilin-1 Ab (1:200) and streptavidin-QD625 (1:100); 5) anti-p-c-Met Ab (1:120) and streptavidin-QD655 (1:100); 6) anti-RANKL Ab (1:100) and streptavidin-QD705 (1:100). All the primary Abs were incubated at 4° C., overnight and streptavidin-QD's reacted at 37° C., 1 hour. After 4 rinses with PBS-Triton (0.4%), the specimens were stained with DAPI and mounted. For negative control, primary Abs were replaced with isotype- and species-matched control Abs and applied to the immediate adjacent tissue sections from 5 pairs of tissue specimens from the studied cases. mQDL was performed in parallel with the tissue slide labeled with the testing primary Abs. The average cell-based intensity from the negative controls was subtracted from the test Ab labeling.

Image Capturing and Biomarker Expression Intensity Quantification

Multiplexed spectral imaging analyses including image acquisition and deconvolution using Nuance 3.0 software and signal quantification using inForm 1.3 software were performed as described in the inventors' previous report [6].

Data Description

The primary outcome is defined as overall survival. Variables measured were Gleason score, race (Caucasian-Americans, African-Americans, and Chinese), and cell-based biomarker expression intensity in cytoplasm, C; nucleus, N; and cytoplasm plus nucleus, C+N of HIF-1α, p-p65, VEGF, NRP-1, p-c-Met and RANKL. Biomarker measurements for each patient were averaged from 4-5 images captured from each of the tumor tissue sites on the slide. An average of 27 images/tissue slide was taken (a range of 4-114 images) which break down to: Caucasian-Americans, 5-114 images; African-Americans, 5-57 images; Chinese, 4-46 images. The total sample size of this study is 54 patients (Caucasian-American, N=20; African-American, N=20; Chinese, N=14). The averaged numbers of cells analyzed with minimum to maximum and standard deviation (SD) are: Caucasian-Americans, 17,207 (1,363-50,488, SD=14,702); African-Americans, 12,541 (2,062-24,612; SD=6,899); Chinese, 3,290 (794-14,770, SD=3,531).

Statistical Analysis

The Kaplan and Meier method was used to estimate overall survival and the logrank test to compare groups. Multivariable proportional hazards regression using forward variable selection was used to assess which biomarkers are predictive of overall survival in the presence of covariates. Proportional hazards assumption was evaluated graphically and analytically, and martingale residuals were used to ensure that the models are appropriate. Critical significance level was set to 5%.

Results

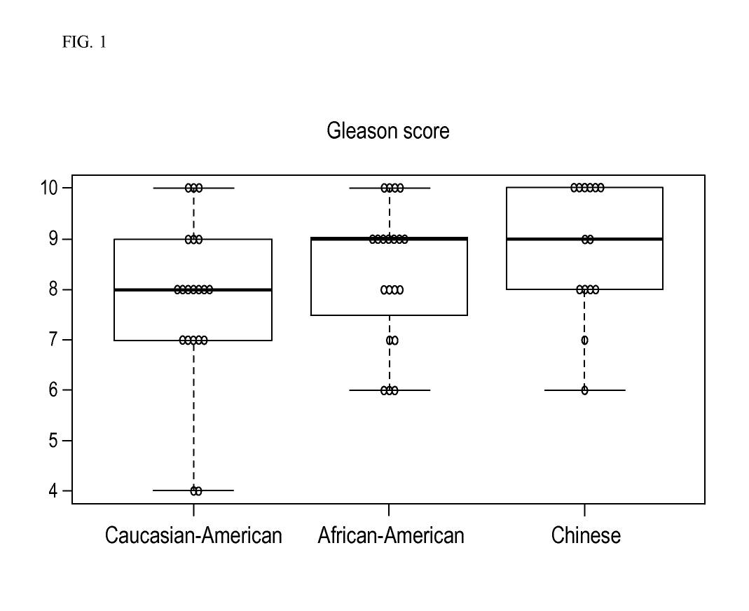

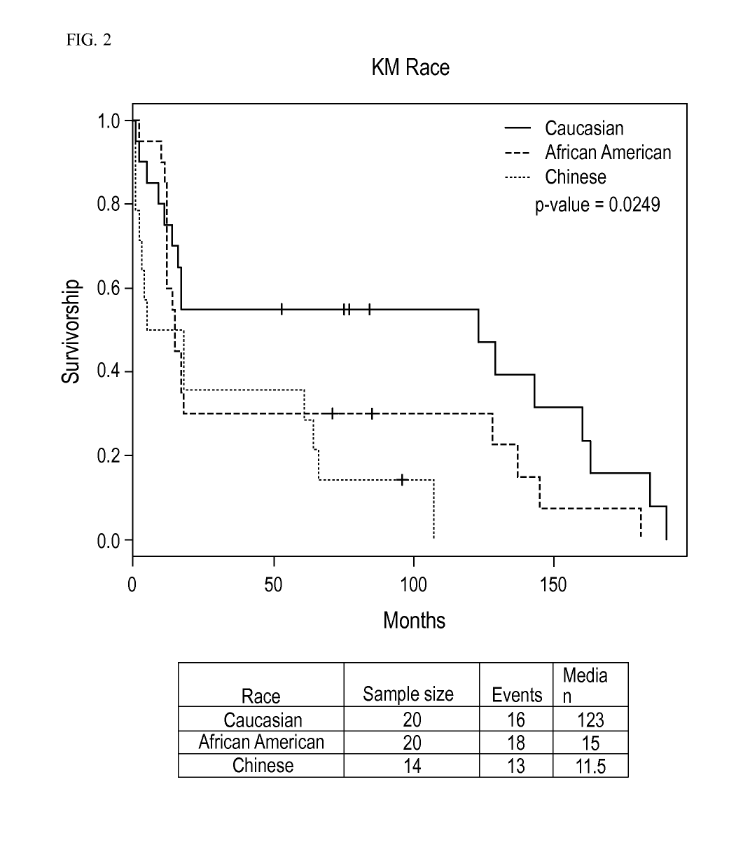

Gleason score box-plots by race among the 3 studied patient groups showed clustered high Gleason scores in Caucasian-Americans, African-Americans and Chinese PC patients (FIG. 1). FIG. 2 shows a significant difference in overall survival by race including all cases (N=54, number of events=47) by Log-rank test (p=0.0249). Furthermore, there was significant difference in biomarkers' mean and standard deviations for combined sample and by each race (Table 1) where Chinese differ from both Caucasian-Americans and African-Americans.

TABLE 1
Biomarker summary statistics for combined sample and by race

| | | Standard | Mean | | | Standard deviation | | |
|---|---|---|---|---|---|---|---|---|
| | Mean | deviation | Caucasian- | African- | | Caucasian- | African- | |
| | All | All | American | American | Chinese | American | American | Chinese |
| Biomarker | (N = 54) | (N = 54) | (N = 20) | (N = 20) | (N = 14) | (N = 20) | (N = 20) | (N = 14) |
| HIF1-α | 13.23 | 14.24 | 14.24 | 20.84 | 0.91 | 10.55 | 16.70 | 1.34 |
| (C + N) | | | | | | | | |
| RANKL | 5.89 | 8.04 | 6.80 | 8.67 | 0.63 | 6.80 | 10.17 | 1.38 |
| (C + N) | | | | | | | | |
| p-p65 | 0.95 | 1.71 | 1.00 | 1.08 | 0.67 | 1.43 | 1.94 | 1.80 |
| (C + N) | | | | | | | | |
| VEGF | 6.14 | 8.61 | 6.63 | 8.28 | 2.40 | 6.34 | 11.18 | 6.20 |
| (C + N) | | | | | | | | |
| NRP-1 | 3.24 | 5.10 | 3.51 | 4.20 | 1.47 | 4.55 | 6.50 | 3.03 |
| (C + N) | | | | | | | | |
| p-c-Met | 5.42 | 6.08 | 5.93 | 7.28 | 2.04 | 5.58 | 6.10 | 5.71 |
| (C + N) | | | | | | | | |
| HIF1-α (N) | 8.15 | 8.86 | 8.62 | 12.98 | 0.60 | 6.39 | 10.52 | 0.94 |
| RANKL (N) | 3.88 | 5.00 | 4.31 | 5.85 | 0.46 | 4.05 | 6.31 | 1.00 |
| p-p65 (N) | 0.44 | 0.81 | 0.47 | 0.51 | 0.29 | 0.70 | 0.96 | 0.77 |
| VEGF (N) | 4.04 | 5.38 | 4.23 | 5.68 | 1.42 | 3.83 | 7.07 | 3.43 |
| NRP-1 (N) | 1.91 | 2.90 | 2.03 | 2.63 | 0.71 | 2.53 | 3.77 | 1.40 |
| p-c-Met (N) | 3.15 | 3.46 | 3.28 | 4.48 | 1.07 | 2.83 | 3.79 | 2.96 |
| HIF1-α (C) | 5.09 | 5.65 | 5.64 | 7.87 | 0.31 | 4.62 | 6.50 | 0.54 |
| RANKL (C) | 2.03 | 3.12 | 2.50 | 2.86 | 0.18 | 2.84 | 3.94 | 0.38 |
| p-p65 (C) | 0.52 | 0.91 | 0.54 | 0.58 | 0.39 | 0.74 | 1.00 | 1.03 |
| VEGF (C) | 2.11 | 3.40 | 2.40 | 2.60 | 0.99 | 2.69 | 4.29 | 2.79 |
| NRP-1 (C) | 1.35 | 2.24 | 1.51 | 1.60 | 0.76 | 2.04 | 2.77 | 1.63 |
| p-c-Met (C) | 2.30 | 2.73 | 2.67 | 2.86 | 0.98 | 2.84 | 2.40 | 2.75 |

C = Cytoplasm;
N = Nucleus

To identify potential biomarkers that predict overall survival of patients with PC, the inventors then analyzed all data independently for each race. Continuous analyses by univariate proportional hazard regression models with Gleason score and biomarkers for Caucasian-American patients (Table 2) showed that RANKL and NRP-1 expression in cytoplasm (C) plus nucleus (N) were significantly correlated with the overall survival of patients with PC, p-value=0.0053 and 0.0029, respectively. Significant associations were also found when expression in C or N was analyzed separately.

TABLE 2
Univariate proportional hazard regression models with
Gleason score and biomarkers for Caucasian-Americans
(N = 20, number of events = 16).

| | | Hazard | |
|---|---|---|---|
| Covariate | Coefficient | ratio | p-value |
| Gleason score | 0.468 | 1.6 | 0.075 |
| HIF1-α (C + N) | 0.0436 | 1.04 | 0.14 |
| RANKL (C + N) | 0.146 | 1.16 | 0.0053 |
| p-p65 (C + N) | 0.197 | 1.22 | 0.21 |
| VEGF (C + N) | 0.0441 | 1.05 | 0.25 |
| NRP-1 (C + N) | 0.197 | 1.22 | 0.0029 |
| p-c-Met (C + N) | −0.0218 | 0.978 | 0.67 |
| HIF1-α (N) | 0.085 | 1.09 | 0.091 |
| RANKL (N) | 0.247 | 1.28 | 0.0072 |
| p-p65 (N) | 0.349 | 1.42 | 0.27 |
| VEGF (N) | 0.0809 | 1.08 | 0.21 |
| NRP-1 (N) | 0.346 | 1.41 | 0.0039 |
| p-c-Met (N) | −0.0337 | 0.967 | 0.74 |
| HIF1-α (C) | 0.0593 | 1.06 | 0.3 |
| RANKL (C) | 0.299 | 1.35 | 0.0058 |
| p-p65 (C) | 0.436 | 1.55 | 0.16 |
| VEGF (C) | 0.0826 | 1.09 | 0.34 |
| NRP-1 (C) | 0.446 | 1.56 | 0.0024 |
| p-c-Met (C) | −0.0513 | 0.95 | 0.62 |

C = Cytoplasm; N = Nucleus

Similar analyses showed that NRP-1 in C, N, and C+N; p-p65 in C, C+N; and VEGF in C were significantly correlated with the overall survival of Chinese patients with PC (Table 3).

TABLE 3
Univariate proportional hazard regression models with
patient Gleason score and biomarkers for Chinese
(N = 14, number of events = 13).

| | | Hazard | |
|---|---|---|---|
| Covariate | Coefficient | ratio | p-value |
| Gleason score | 0.45 | 1.58 | 0.076 |
| HIF1-α (C + N) | −0.0788 | 0.924 | 0.74 |
| RANKL (C + N) | 0.0589 | 1.06 | 0.76 |
| p-p65 (C + N) | 0.362 | 1.44 | 0.049 |
| VEGF (C + N) | 0.104 | 1.11 | 0.054 |
| NRP-1 (C + N) | 0.248 | 1.28 | 0.033 |
| p-c-Met (C + N) | −0.00726 | 0.993 | 0.87 |
| HIF1-α (N) | 0.0982 | 1.1 | 0.78 |
| RANKL (N) | 0.0758 | 1.08 | 0.77 |
| p-p65 (N) | 0.823 | 2.28 | 0.056 |
| VEGF (N) | 0.182 | 1.2 | 0.067 |
| NRP-1 (N) | 0.51 | 1.67 | 0.042 |
| p-c-Met (N) | −0.0136 | 0.987 | 0.88 |
| HIF1-α (C) | −0.587 | 0.556 | 0.35 |
| RANKL (C) | 0.255 | 1.29 | 0.72 |
| p-p65 (C) | 0.648 | 1.91 | 0.044 |
| VEGF (C) | 0.238 | 1.27 | 0.046 |
| NRP-1 (C) | 0.47 | 1.6 | 0.028 |
| p-c-Met (C) | −0.0156 | 0.985 | 0.87 |

C = Cytoplasm; N = Nucleus

FIG. 3 shows NRP-1, p-p65 and VEGF protein expression images from the mQDL of tissues obtained from a Chinese patient who survived for 66 months (long) vs a patient who survived for 2 months (short). In contrast, with the exception of Gleason score (p<0.027), none of the 6 biomarkers reached significant association with survival time of African-American patients analyzed by the same method (Table 4).

TABLE 4
Univariate proportional hazard regression models with
patient Gleason score and biomarkers for
African-Americans (N = 20, number of events = 18).

| | | Hazard | |
|---|---|---|---|
| Covariate | Coefficient | ratio | p-value |
| Gleason score | 0.534 | 1.71 | 0.027 |
| HIF1-α (C + N) | 0.0131 | 1.01 | 0.3 |
| RANKL (C + N) | 0.0124 | 1.01 | 0.56 |
| p-p65 (C + N) | −0.102 | 0.903 | 0.42 |
| VEGF (C + N) | −0.0158 | 0.984 | 0.44 |
| NRP-1 (C + N) | −0.0204 | 0.98 | 0.58 |
| p-c-Met (C + N) | 0.0658 | 1.07 | 0.091 |
| HIF1-α (N) | 0.0202 | 1.02 | 0.31 |
| RANKL (N) | 0.0239 | 1.02 | 0.49 |
| p-p65 (N) | −0.25 | 0.779 | 0.34 |
| VEGF (N) | −0.0167 | 0.983 | 0.6 |
| NRP-1 (N) | −0.0226 | 0.978 | 0.72 |
| p-c-Met (N) | 0.116 | 1.12 | 0.06 |
| HIF1-α (C) | 0.0336 | 1.03 | 0.31 |
| RANKL (C) | 0.0227 | 1.02 | 0.69 |
| p-p65 (C) | −0.155 | 0.856 | 0.52 |
| VEGF (C) | −0.0639 | 0.938 | 0.28 |
| NRP-1 (C) | −0.0686 | 0.934 | 0.43 |
| p-c-Met (C) | 0.144 | 1.15 | 0.15 |

C = Cytoplasm;
N = Nucleus

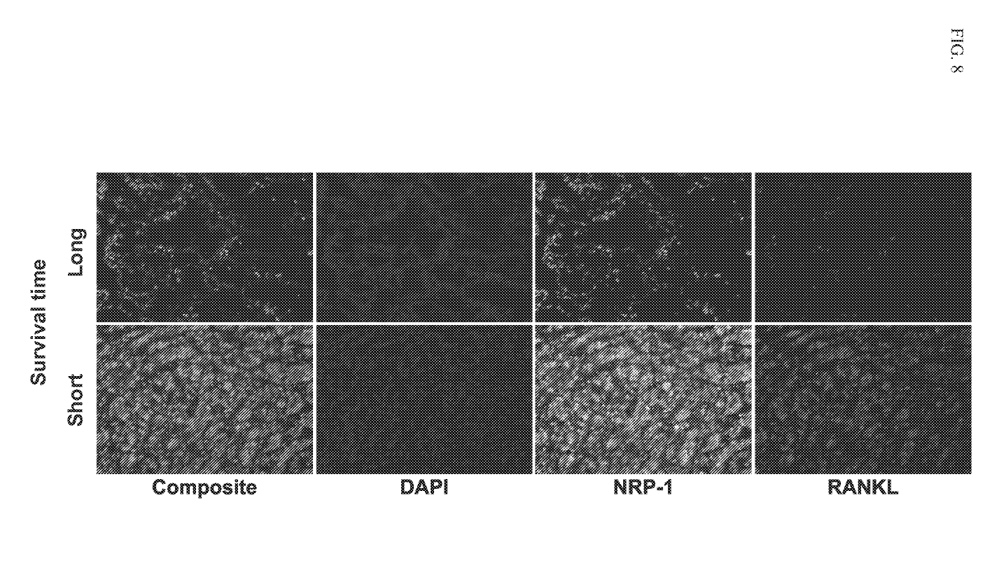

Correlograms (FIGS. 4-6) showed pair-wise correlations between biomarkers with each other, and biomarkers with Gleason scores among Caucasian-Americans, African-Americans and Chinese patients, respectively. The main diagonal shows the covariate names for each pair-wise comparison. The center at the horizontal and vertical interaction of each covariate is the Pearson correlation coefficient and at the top right is the associated p value. Results showed that there were significant correlations between most of the biomarker pairs (in bold) irrespective of the race but only HIF-1α correlates with Gleason score for Caucasian-American patients, p=0.002. FIG. 7 shows additional discretized visualizations of the effect of categorized biomarkers on overall survival of the Caucasian patients as analyzed by Kaplan and Meier method and log-rank test to compare biomarker protein expression in cytoplasm plus nucleus categorized in two groups, high and low, using the median as a cutoff point. RANKL and NRP-1 correlated significantly with overall survival, with p-value=0.025 and 0.005, respectively. FIG. 8 presents unmixed mQDL images of NRP-1 and RANKL expression from representative tissues from a Caucasian-American patient who survived for 163 months (long) vs. a patient who survived only 2 months (short). Similar analyses performed in African-American and Chinese patients did not show a correlation between RANKL and NRP-1 biomarkers and patient overall survival (data not included). For African-Americans, although only Gleason scores were significant in the univariate model (Table 4), nuclear p-c-Met became a significant predictor in combination with Gleason score (Table 5) in a multivariable proportional hazard regression model (p<0.025 and p<0.044, respectively).

TABLE 5
Multivariable proportional hazard regression models with patient
Gleason score, nuclear p-c-Met, after variable selection,
for African-Americans (N = 20, number of events = 18).

| | | | | Null martingale |
|---|---|---|---|---|
| | | Hazard | | residual analysis |
| Covariate | Coefficient | ratio | p-value | (p-value) |
| Gleason score | 0.611 | 1.84 | 0.025 | 0.129 |
| Nuclear p-c-Met | 0.139 | 1.15 | 0.044 | 0.445 |
| (continuous) | | | | |

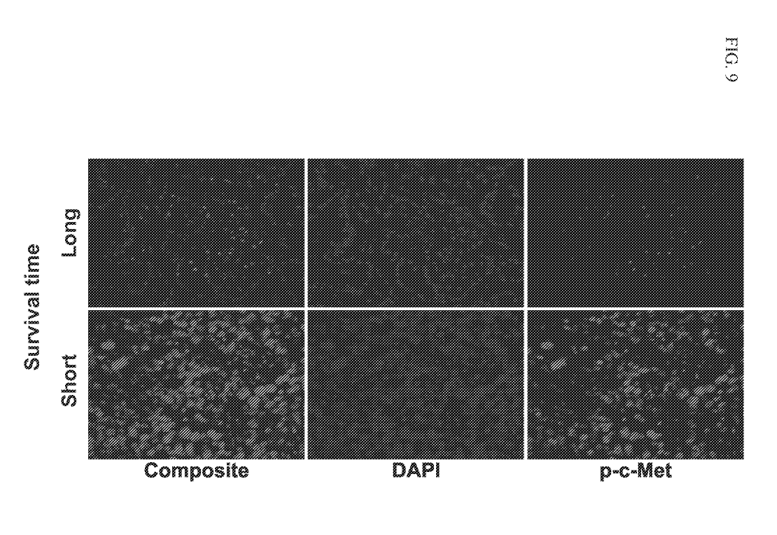

FIG. 9 shows the unmixed mQDL images of p-c-Met protein expression in an African-American patient who survived for 85 months (long) vs an African-American patient who survived for 12 months (short). To visualize the effect of these two variables on overall survival, Gleason score was categorized into two groups: >8 and <8, and nuclear p-c-Met was categorized in two groups, high and low, using the median as a cutoff point (FIG. 10, p-value=0.0349).

TABLE 10
Data in Figure 10

| Group | Sample size | Events | Median |
|---|---|---|---|
| Gleason ≧ 8, Biomarker Low | 6 | 5 | 16 |
| Gleason ≧ 8, Biomarker High | 9 | 9 | 12 |
| Gleason < 8, Biomarker Low | 4 | 3 | 137 |
| Gleason < 8, Biomarker High | 1 | 1 | 12 |

To further explore in a systematic way whether combining Gleason score and a biomarker may improve the prediction of overall survival, all biomarkers that were significant predictors of overall survival in univariate models from Tables 2-4 were categorized in two groups, high and low, using the median as a cutoff point. Gleason score was categorized into two groups: >8 and <8 and the two dichotomous variables were combined to generate four groups: Gleason>8 and Biomarker High, Gleason ≧8 and Biomarker Low, Gleason <8 and Biomarker High, and Gleason <8 and Biomarker Low. Three dummy variables were created, using the group Gleason <8 and Biomarker Low as the reference, and multivariable proportional hazards regression using forward variable selection was used to select the best model to predict overall survival. The results of these multivariable models are shown in Table 6.

TABLE 6
Multivariable proportional hazards regression
using forward variable selection was used to
select the best model to predict overall survival.

| | | Categorized |
|---|---|---|
| | | Gleason/Biomarker |
| | | group Predictive |
| Population | Biomarker | of overall survival |
| Caucasian-American | RANKL (C + N) | none |
| Caucasian-American | NRP-1 (C + N) | none |
| Caucasian-American | RANKL (N) | none |
| Caucasian-American | NRP-1 (N) | none |
| Caucasian-American | RANKL (C) | none |
| Caucasian-American | NRP-1 (C) | none |
| African-American | p-c-Met (C + N) | none |
| African-American | p-c-Met (N) | [Gleason >= 8, |
| | | Biomarker High] |
| Chinese | p-p65 (C + N) | none |
| Chinese | NRP-1 (C + N) | none |
| Chinese | NRP-1 (N) | none |
| Chinese | p-p65 (C) | none |
| Chinese | VEGF (C) | none |
| Chinese | NRP-1 (C) | none |

C = Cytoplasm;
N = Nucleus

All biomarkers that were significant predictors of overall survival in univariate models from Tables 2-4 were categorized in two groups, high and low, using the median as a cutoff point. Gleason score was categorized into two groups: ≧8 and <8 and the two dichotomous variables were combined to generate four groups: Gleason ≧8 and Biomarker High, Gleason ≧8 and Biomarker Low, Gleason <8 and Biomarker High, and Gleason <8 and Biomarker Low. ‘Biomarker High’ indicates biomarker values greater than the median of the (continuous) biomarker.

While there was no categorized Gleason/Biomarker group predictive of overall survival for Caucasian-Americans and Chinese, for African-Americans the results with the categorized groups agreed with the multivariable continuous predictor model and details of the analysis as shown in Table 7.

TABLE 7
Multivariable proportional hazard regression models
with all binary dummy variables for African-American
(N = 20, number of events = 18).

| | | | | Null martingale |
|---|---|---|---|---|
| Binary | | Hazard | | residual analysis |
| dummy variable | Coefficient | ratio | p-value | (p-value) |
| Gleason ≧ 8, | 1.924 | 6.85 | 0.015 | 0.5 |
| Biomarker High | | | | |
| Gleason ≧ 8, | 0.622 | 1.86 | 0.42 | 0.06 |
| Biomarker Low | | | | |
| Gleason < 8, | 2.49 | 12.06 | 0.052 | 0.71 |
| Biomarker High | | | | |

‘Biomarker High’ indicates biomarker values above the median of the (continuous) biomarker. ‘Biomarker Low’ indicates biomarker values below or equal to the median of the (continuous) biomarker.

The final model is shown in Table 8a and displayed in FIG. 11 where only “Gleason ≧8/Biomarker High” is a significant predictor of overall survival in African American patients with prostate cancer (p<0.0117).

TABLE 8a
Multivariable proportional hazard regression model,
with significant binary dummy variable for
African-Americans (N = 20, number of events = 18).

| | | | | Null martingale |
|---|---|---|---|---|
| Binary | | Hazard | | residual analysis |
| dummy variable | Coefficient | ratio | p-value | (p-value) |
| Gleason ≧ 8, | 1.34 | 3.83 | 0.019 | 0.395 |
| Biomarker High | | | | |

‘Biomarker High’ indicates biomarker values above the median of the (continuous) biomarker.

TABLE 8b
Data for Figure 11.

| Group | Sample size | Events | Median |
|---|---|---|---|
| Gleason ≧ 8, Biomarker High | 9 | 9 | 12 |
| Not(Gleason ≧ 8, Biomarker High) | 11 | 9 | 128 |

### Example 2

### RANKL Predicts Prostate Cancer Bone Metastasis and Lethal Phenotype of Human Prostate Cancer

The inventors found RANKL expression in primary human prostate cancer predicts human prostate cancer survival in patients. These results supported and validated the animal model described herein.

The graph (FIG. 7) shown represent the results obtained from 20 patients with their survival either low or high with about equal distribution. Each of the patients had 2-14 tissue specimens from TURP and are subjected to immunohistochemistry staining with RANKL antibody. RANKL antibody detects RANKL protein expression in these studies. After staining the tissue specimens with anti-RANKL antibody, the inventors evaluated an average of greater than 12,000 single cells and evaluated RANKL distribution in cytosol, cell membrane, and nucleus. By multiplexing quantum dot using an automated Vectra imaging system, the data plotted is RANK order of intensity directly read from the imaging analyzing system. These series of data was then analyzed in a double blind manner. The plot revealed RANKL is a significant biomarker that can differentiate patients with either long (over 100 months) or short survival. In the same assay, the inventors observed that HIF-1α, phosphorylated NF-κB, VEGF, and phosphorylated c-Met showed no correlation with survival of prostate cancer patients.

### Example 3

### Circulating Tumor Cells (CTC) Detection of CTC with Fluorescence Activated Cell Sorting (FACS)

Two FACS instruments (BD Biosciences, MA) were used in the study. To isolate CTCs, a FACSAria III was used to sort positively labeled cells onto an APES-coated cytology slide (Bio-World, Dublin, Ohio). To enumerate CTCs, an LSRII Flow Cytometer was used. Manufacturer recommended detection procedures were followed. In parallel to the detection of each human blood sample, 1×10⁴ PC-3 cells were used to spike a 1 ml aliquot of the sample. The flow cytometric profile of the spiked sample was used to guide the positivity gating. FACS data was further analyzed with FlowJo software.

### Fluorescence Imaging

Stained cells isolated with the FACS sorter on slides were subjected to both fluorescence imaging and near infrared imaging, with a Nikon Eclipse Ti fluorescence microscope excited by a xenon arc light source. Near infrared images were acquired through an INDO filter (780-840 nm).

Multiple Quantum Dot Labeling (mQDL)

Stained cells collected with the FACS sorter on glass slides were subjected to further staining with the mQDL protocol as previously reported [Hu P, Chu G C, Zhu G, Yang H, Luthringer D, et al. (2011) Multiplexed quantum dot labeling of activated c-Met signaling in castration-resistant human prostate cancer. PLoS One 6: e28670]. In brief, the samples were first treated with stripping buffer to remove the mAb used for CTC isolation, and then subjected to successive staining with antibodies reacting to a group of PCa-related biomarkers, including RANKL, HIF-1α, NRP-1, VEGF, p-c-MET, and p-p65, as previously reported [Hu et al. 2011], with the same staining protocol. Finally, the samples were counterstained with DAPI before being subjected to spectral imaging and signal quantification on a CRi spectral imaging system with Nuance software (Caliper Life Sciences, Hopkinton, Mass.).

### Isolation of Live CTCs for Further Molecular Characterization

NIR staining facilitated the identification and isolation of live CTCs from clinical blood samples. We tested the isolated CTCs to further investigate PCa-related molecular alterations. In one such investigation, CTCs in PCa patients were isolated based on EpCAM⁺ CD45⁻NIR⁺DAPI+ staining. The gene expression profiles of CTCs on the microscopic slides were detected by mQDL to determine if a panel of protein biomarkers stained by quantum dots could be associated with PCa progression and metastasis. These assays demonstrated that the abnormal expression of RANKL, HIF-1α, NRP-1 and VEGF proteins seen in clinical PCa tumor specimens could be easily detected in the isolated CTCs (FIG. 15). Similarly to clinical tumors, enhanced phosphorylation of c-Met, as well as the p65 subunit of the NFκB, was detected in the same CTC population. Intriguingly, signal quantification of the stained CTCs revealed remarkable intercellular heterogeneity, as individual proteins were detected with varied levels among CTCs (FIG. 15).

The isolated live CTCs were amenable to multiplex detection of protein levels at the single cell level in freshly isolated CTCs using a mQDL method (FIG. 15). This shows that isolated CTCs are appropriate for biological analysis such as mQDL analysis for protein expression at the single cell level.

TABLE 9

| | | Clinical | | | | Live |
|---|---|---|---|---|---|---|
| patient | Day of | information | PSA | Total | NIR + | CTC |
| ID | analysis | Therapy | (ng/ml) | CTC/ml | CTC/ml | (%) |
| 44 | 0 | On bi- | 3.3 | 1052 | 922 | 88% |
| | | calutamide | | | | |
| | 44 | Stopping bi- | 7.3 | 54 | 35 | 65% |
| | | calutamide | | | | |
| | 25 | Developing | 8.5 | 196 | 123 | 63% |
| | | shoulder | | | | |
| | | pain | | | | |

### Example 4

Prostate cancer tumor cells, LNCaP cells transfected with RANKL known to develop high incidence of metastases to bone and soft tissues (Hu, et al. Multiplexed quantum dot labeling of activated c-Met signaling in castration-resistant human prostate cancer. Plos one, 6: e28670, 2011), were implanted in either sham-operated control of surgically-castrated mice (androgen deprivation) and mice either fed with control diet or fed with high cholesterol diet. As seen in FIG. 17, pathophysiological conditions elevating RANKL in castrated mice and in mice fed with high cholesterol diet had increased incidence and cancer bone and soft tissue metastases. The incidence of cancer metastases to bone and soft tissues was also correlated with the number of CTCs harvested from the mice.

### Example 5

Experiments based on 44 cases allowed the inventors to also analyze metastasis and castration resistance. The inventors analyzed the protein expression in nucleus (N), cytoplasm (C) and both nucleus plus cytoplasm (N+C). Statistical analyses were performed for overall survival, metastasis (Mets) and castration resistance (CR) correlation.

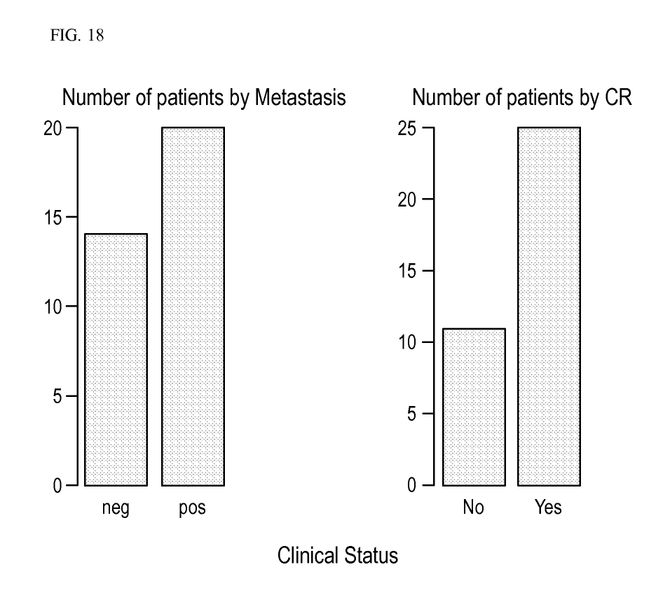

Metastasis and Castration Resistance status of the patients from whom the specimens were obtained (FIG. 18).

For the data below, since the expressions are skewed, a log transformation was used for all the protein expression and then used in the logistic regression. The following tables show the result of logistic regression. It's a univariate model because those expressions are highly correlated.

$$y = logit ( p ) = ln ( \frac{p}{1 - p} ) = X β$$

where, X is the protein expression, p is the probability of Yes (for CR) or Pos(for Metastasis), β=coefficient; β0=intercept; β1=slope; *=times; ln is log use e as the base;

Since p is a probability, it can only be (0,1), it violates the normal assumption in regular linear regression, so we use a transformation of p as the dependent variable Log(p/(1−p))=(β0+β1)*X.

For the tables below: X=variables (protein expression); Estimate=β1; Intercept=β0; P in the last column on the right is the p value for the model.

Cancer cell: p-c-Met (C), RANKL (N, C, N+C), NRP1 (N,C, N+C) correlate with castration resistance (Tables 10 and 11).

TABLE 10
Univariate logistic regression results for Metastasis

| | variable | Estimate | StdError | z | p |
|---|---|---|---|---|---|
| 1 | Nucleus_565_pc_Met | −0.0479 | 0.3578 | −0.1338 | 0.8936 |
| 2 | Nucleus_585_RANKL | 0.5371 | 0.5258 | 1.0214 | 0.3071 |
| 3 | Nucleus_605_RANK | 0.3614 | 0.2886 | 1.2521 | 0.2104 |
| 4 | Nucleus_625_NRP1 | 0.3204 | 0.2451 | 1.3069 | 0.1912 |
| 5 | Cytoplasm_565_pc_Met | 0.2341 | 0.3096 | 0.7560 | 0.4497 |
| 6 | Cytoplasm_585_RANKL | 0.5115 | 0.4593 | 1.1136 | 0.2654 |
| 7 | Cytoplasm_605_RANK | 0.3712 | 0.3638 | 1.0203 | 0.3076 |
| 8 | Cytoplasm_625_NRP1 | 0.3634 | 0.2708 | 1.3418 | 0.1797 |
| 9 | Total_565_pc_Met | 0.0520 | 0.3440 | 0.1510 | 0.8800 |
| 10 | Total_585_RANKL | 0.5345 | 0.5001 | 1.0688 | 0.2852 |
| 11 | Total_605_RANK | 0.3779 | 0.3498 | 1.0804 | 0.2800 |
| 12 | Total_625_NRP1 | 0.3528 | 0.2620 | 1.3466 | 0.1781 |

TABLE 11
Univariate logistic regression results for CR

| | variable | Estimate | StdError | z | p |
|---|---|---|---|---|---|
| 1 | Nucleus-565_pc_Met | 0.6297 | 0.4400 | 1.4311 | 0.1524 |
| 2 | Nucleus-585-RANKL | 1.1974 | 0.5731 | 2.0895 | 0.0367 |
| 3 | Nucicus-605_RANK | 0.4190 | 0.2807 | 1.4930 | 0.1354 |
| 4 | Nucleus_625_NRP1 | 0.5545 | 0.2611 | 2.1233 | 0.0337 |
| 5 | Cytoplasm_565_pc_Met | 0.8862 | 0.4439 | 1.9964 | 0.0459 |
| 6 | Cytoplasm_585_RANKL | 1.2499 | 0.5322 | 2.3488 | 0.0188 |
| 7 | Cytoplasm_605_RANK | 0.6178 | 0.3714 | 1.6634 | 0.0962 |
| 8 | Cytoplasm_625_NRP1 | 0.6908 | 0.3035 | 2.2764 | 0.0228 |
| 9 | Total_565_pc_Met | 0.7189 | 0.4403 | 1.6327 | 0.1025 |
| 10 | Total_585_RANKL | 1.2638 | 0.5644 | 2.2392 | 0.0251 |
| 11 | Total_605_RANK | 0.5820 | 0.3548 | 1.6405 | 0.1009 |
| 12 | Total_625_NRP1 | 0.6411 | 0.2873 | 2.2318 | 0.0256 |

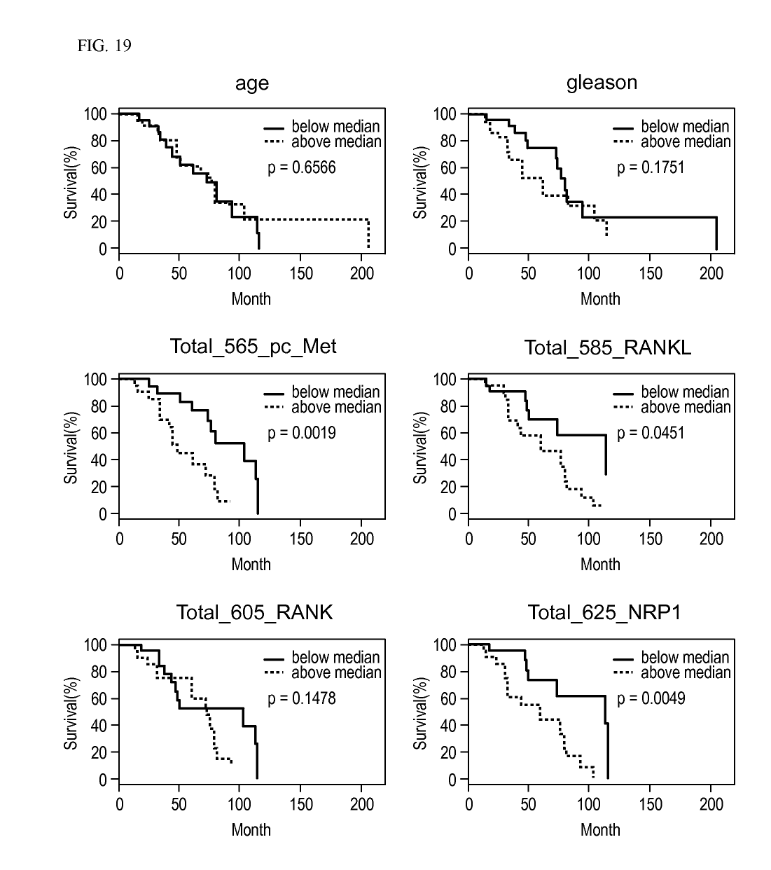

Cancer-associated stroma: P-c-Met (N+C), RANKL (N+C), NRP1 N+C) expression correlate with overall survival (FIG. 19). p-c-Met (C), RANKL (N, C, N+C), NRP1 (N,C, N+C) correlate with castration resistance (Tables 12 and 13).

TABLE 12
Univariate logistic regression results for Metastasis

| | variable | Estimate | StdError | z | p |
|---|---|---|---|---|---|
| 1 | Nucleus_565_pc_Met | −0.0008 | 0.3640 | −0.0022 | 0.9982 |
| 2 | Nucleus_585_RANKL | 1.0576 | 0.7535 | 1.4037 | 0.1604 |
| 3 | Nucleus_605_RANK | 0.3421 | 0.3244 | 1.0546 | 0.2916 |
| 4 | Nucleus_625_NRP1 | 0.4731 | 0.3158 | 1.4983 | 0.1341 |
| 5 | Cytoplasm_565_pc_Met | 0.2317 | 0.3299 | 0.7023 | 0.4825 |
| 6 | Cytoplasm_585_RANKL | 0.7299 | 0.5783 | 1.2622 | 0.2069 |
| 7 | Cytoplasm_605_RANK | 0.2851 | 0.4621 | 0.6169 | 0.5373 |
| 8 | Cytoplasm_625_NRP1 | 0.5590 | 0.3612 | 1.5474 | 0.1218 |
| 9 | Total_565_pc_Met | 0.0679 | 0.3566 | 0.1905 | 0.8489 |
| 10 | Total_585_RANKL | 0.9262 | 0.6806 | 1.3609 | 0.1735 |
| 11 | Total_605-RANK | 0.3118 | 0.4367 | 0.7140 | 0.4752 |
| 12 | Total_625_NRP1 | 0.5322 | 0.3445 | 1.5449 | 0.1224 |

TABLE 13
Univariate logistic regression results for CR

| | variable | Estimate | StdError | z | p |
|---|---|---|---|---|---|
| 1 | Nucleus_565_pc_Met | 0.7112 | 0.4510 | 1.5771 | 0.1148 |
| 2 | Nucleus_585_RANKL | 1.7256 | 0.8584 | 2.0102 | 0.0444 |
| 3 | Nucleus_605_RANK | 0.3531 | 0.3070 | 1.1500 | 0.2501 |
| 4 | Nucleus_625_NRP1 | 0.6986 | 0.3274 | 2.1339 | 0.0328 |
| 5 | Cytoplasm_565_pc_Met | 0.8893 | 0.4471 | 1.9891 | 0.0467 |
| 6 | Cytoplasm_585_RANKL | 1.5393 | 0.6753 | 2.2793 | 0.0226 |
| 7 | Cytoplasm_605_RANK | 0.5651 | 0.4397 | 1.2852 | 0.1987 |
| 8 | Cytoplasm_625_NRP1 | 0.9400 | 0.4036 | 2.3290 | 0.0199 |
| 9 | Total_565_pc_Met | 0.7724 | 0.4519 | 1.7093 | 0.0874 |
| 10 | Total_585_RANKL | 1.7283 | 0.7935 | 2.1781 | 0.0294 |
| 11 | Total_605_RANK | 0.5301 | 0.4152 | 1.2768 | 0.2017 |
| 12 | Total_625_NRP1 | 0.8419 | 0.3722 | 2.2620 | 0.0237 |

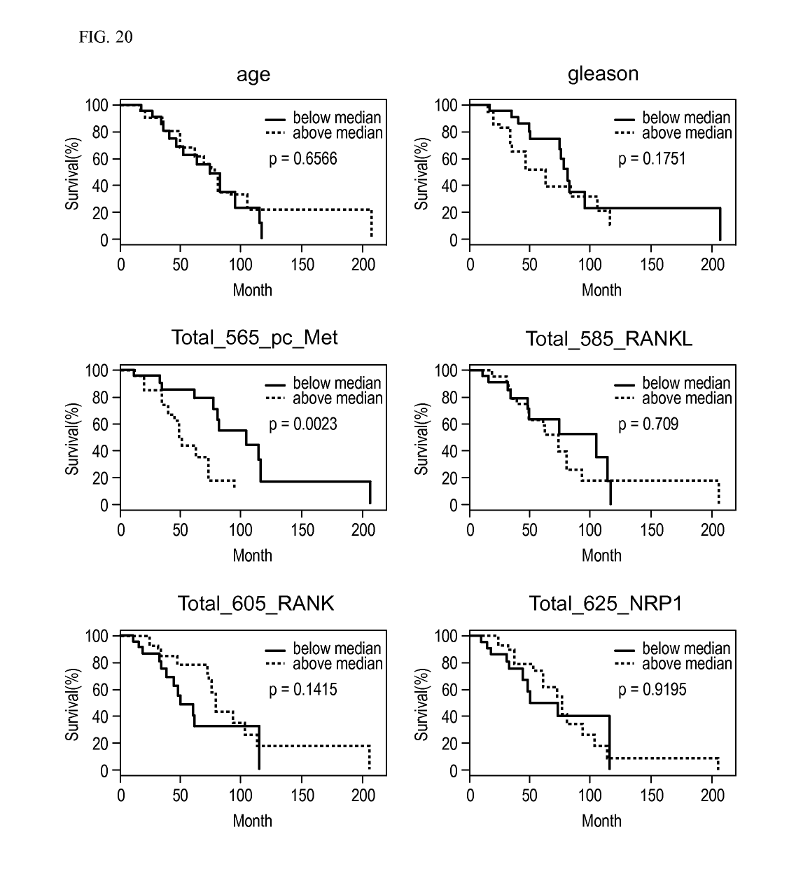

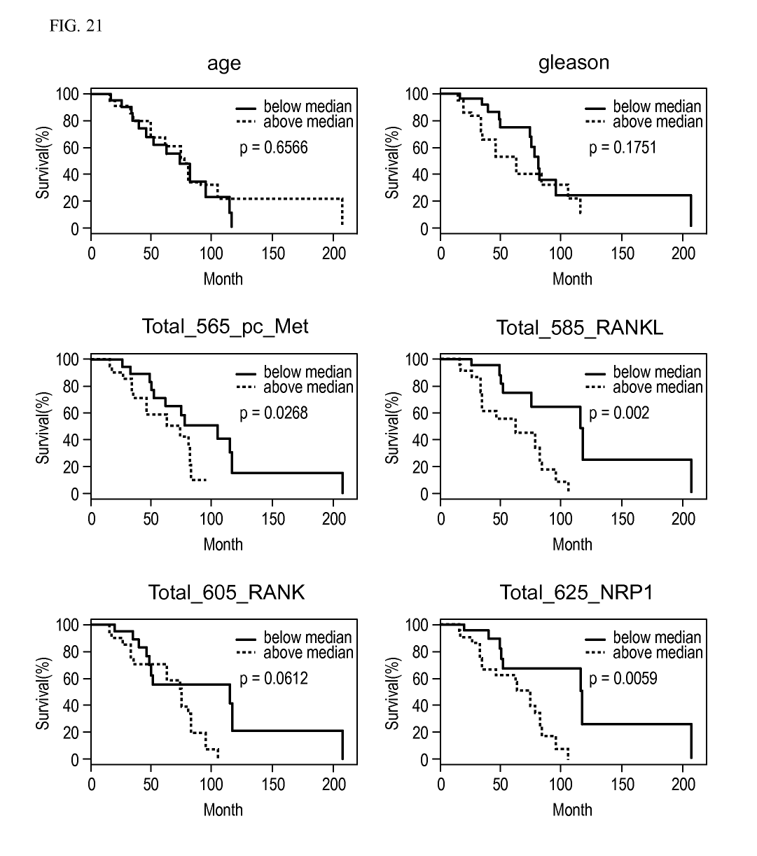

Non-cancer-associated stroma: p-c-Met (N+C) expression correlate with overall survival (FIG. 20). RANK (N) expression correlate with metastasis (Tables 14 and 15).

TABLE 14
Univariate logistic regression results for Metastasis

| | variable | Estimate | StdError | z | p |
|---|---|---|---|---|---|
| 1 | Nucleus_565_pc_Met | −0.2985 | 0.3709 | −0.8049 | 0.4209 |
| 2 | Nucleus_585_RANKL | 0.0570 | 0.9266 | 0.0615 | 0.9510 |
| 3 | Nucleus_605_RANK | 0.6186 | 0.2906 | 2.1290 | 0.0333 |
| 4 | Nucleus_625_NRP1 | 0.3982 | 0.4151 | 0.9592 | 0.3375 |
| 5 | Cytoplasm_565_pc_Met | −0.3845 | 0.3688 | −1.0424 | 0.2972 |
| 6 | Cytoplasm_585_RANKL | −0.2465 | 0.6988 | −0.3527 | 0.7243 |
| 7 | Cytoplasm_605_RANK | 0.5149 | 0.4229 | 1.2175 | 0.2234 |
| 8 | Cytoplasm_625_NRP1 | 0.2274 | 0.4317 | 0.5267 | 0.5984 |
| 9 | Total_565_pc_Met | −0.3141 | 0.3775 | −0.8321 | 0.4051 |
| 10 | Total_585_RANKL | −0.0782 | 0.8621 | −0.0907 | 0.9277 |
| 11 | Total_605_RANK | 0.6162 | 0.1003 | 1.5392 | 0.1238 |
| 12 | Total_625_NRP1 | 0.3236 | 0.4300 | 0.7526 | 0.4517 |

TABLE 15
Univariate logistic regression results for CR

| | variable | Estimate | StdError | z | p |
|---|---|---|---|---|---|
| 1 | Nucleus_565_pc_Met | −0.1063 | 0.3973 | −0.2676 | 0.7890 |
| 2 | Nucleus_585_RANKL | 1.2779 | 1.0209 | 1.2517 | 0.2107 |
| 3 | Nucleus_605_RANK | 0.3220 | 0.2726 | 1.1813 | 0.2375 |
| 4 | Nucleus_625_NRP1 | 0.5385 | 0.4087 | 1.3175 | 0.1877 |
| 5 | Cytoplasm_565_pc_Met | 0.2070 | 0.4008 | 0.5164 | 0.6056 |
| 6 | Cytoplasm_585_RANKL | 0.7599 | 0.6509 | 1.1675 | 0.2430 |
| 7 | Cytoplasm-605_RANK | 0.4003 | 0.4736 | 0.8452 | 0.3980 |
| 8 | Cytoplasm_625_NRP1 | 0.5353 | 0.4154 | 1.2018 | 0.2295 |
| 9 | Total_565_pc_Met | −0.0411 | 0.4010 | −0.1026 | 0.9]83 |
| 10 | Total_585_RANKL | 1.1582 | 0.9042 | 1.2809 | 0.2002 |
| 11 | Total_605_RANK | 0.4065 | 0.4338 | 0.9372 | 0.3486 |
| 12 | Total_625_NRP1 | 0.5547 | 0.4351 | 1.2749 | 0.2024 |

Morphologically Normal Glands: NRP1 (N) expression correlates with castration resistance (Tables 16 and 17).

TABLE 16
Univariate logistic recession results for Metastasis

| | variable | Estimate | StdError | z | p |
|---|---|---|---|---|---|
| 1 | Nucleus_565_pc_Met | −0.4484 | 0.5238 | −0.8559 | 0.3920 |
| 2 | Nucleus_585_RANKL | 1.3958 | 1.0193 | 1.3694 | 0.1709 |
| 3 | Nucleus_605_RANK | 0.4425 | 0.3891 | 1.1373 | 0.2554 |
| 4 | Nucleus_625_NRP1 | 1.0152 | 0.5581 | 1.8190 | 0.0689 |
| 5 | Cytoplasm_565_pc_Met | −0.2218 | 0.4145 | −0.5351 | 0.5926 |
| 6 | Cytoplasm_585_RANKL | 0.6221 | 0.7758 | 0.8019 | 0.4226 |
| 7 | Cytoplasm_605_RANK | 0.6330 | 0.5308 | 1.1926 | 0.2330 |
| 8 | Cytoplasm_625_NRP1 | 0.8340 | 0.5275 | 1.5810 | 0.1139 |
| 9 | Total_565_pc_Met | −0.3570 | 0.4827 | −0.7396 | 0.4595 |
| 10 | Total_585_RANKL | 0.9617 | 0.8901 | 1.0805 | 0.2799 |
| 11 | Total_605_RANK | 0.6500 | 0.5201 | 1.2499 | 0.2113 |
| 12 | Total_625_NRP1 | 0.9212 | 0.5421 | 1.6993 | 0.0893 |

TABLE 17
Univariate logistic regression results for OR

| | variable | Estimate | StdError | z | p |
|---|---|---|---|---|---|
| 1 | Nucleus_565_pc_Met | −0.0850 | 0.4784 | −0.1778 | 0.8589 |
| 2 | Nucleus_585_RANKL | 2.3732 | 1.2558 | 1.8897 | 0.0588 |
| 3 | Nucleus_605_RANK | 0.3995 | 0.3407 | 1.1724 | 0.2410 |
| 4 | Nucleus_625_NRP1 | 0.9453 | 0.4795 | 1.9712 | 0.0487 |
| 5 | Cytoplasm_565_pc_Met | 0.1908 | 0.3956 | 0.4824 | 0.6295 |
| 6 | Cytoplasm_585_RANKL | 1.5845 | 0.9079 | 1.7452 | 0.0810 |
| 7 | Cytoplasm_605_RANK | 0.7335 | 0.5427 | 1.3516 | 0.1765 |
| 8 | Cytoplasm_625_NRP1 | 0.8987 | 0.5065 | 1.7745 | 0.0760 |
| 9 | Total_565_pc_Met | 0.0295 | 0.4456 | 0.0663 | 0.9171 |
| 10 | Total_585_RANKL | 2.0445 | 1.1103 | 1.8113 | 0.0656 |
| 11 | Total_605_RANK | 0.6792 | 0.5128 | 1.3246 | 0.1853 |
| 12 | Total_625_NRP1 | 0.9371 | 0.5008 | 1.8712 | 0.0613 |

### REFERENCES

1. Grivas P D, Robins D M, Hussain M (2013) Predicting response to hormonal therapy and survival in men with hormone sensitive metastatic prostate cancer. Crit Rev Oncol Hematol 85: 82-93.
2. Kachroo N, Gnanapragasam V J (2013) The role of treatment modality on the utility of predictive tissue biomarkers in clinical prostate cancer: a systematic review. J Cancer Res Clin Oncol 139: 1-24.
3. Tran P T, Hales R K, Zeng J, Aziz K, Salih T, et al. (2012) Tissue biomarkers for prostate cancer radiation therapy. Curr Mol Med 12: 772-787.
4. Koeneman K S, Yeung F, Chung L W (1999) Osteomimetic properties of prostate cancer cells: a hypothesis supporting the predilection of prostate cancer metastasis and growth in the bone environment. Prostate 39: 246-261.
5. Josson S, Matsuoka Y, Chung L W, Zhau H E, Wang R (2010) Tumor-stroma co-evolution in prostate cancer progression and metastasis. Semin Cell Dev Biol 21: 26-32.
6. Hu P, Chu G C, Zhu G, Yang H, Luthringer D, et al. (2011) Multiplexed quantum dot labeling of activated c-Met signaling in castration-resistant human prostate cancer. PLoS One 6: e28670.
7. Lue H W, Yang X, Wang R, Qian W, Xu R Z, et al. (2011) LIV-1 promotes prostate cancer epithelial-to-mesenchymal transition and metastasis through HB-EGF shedding and EGFR-mediated ERK signaling. PLoS One 6: e27720.
8. Odero-Marah V A, Wang R, Chu G, Zayzafoon M, Xu J, et al. (2008) Receptor activator of NF-kappaB Ligand (RANKL) expression is associated with epithelial to mesenchymal transition in human prostate cancer cells. Cell Res 18: 858-870.
9. Xu J, Wang R, Xie Z H, Odero-Marah V, Pathak S, et al. (2006) Prostate cancer metastasis: role of the host microenvironment in promoting epithelial to mesenchymal transition and increased bone and adrenal gland metastasis. Prostate 66: 1664-1673.
10. Zhang S, Wang X, Iqbal S, Wang Y, Osunkoya A O, et al. (2013) Epidermal growth factor promotes protein degradation of epithelial protein lost in neoplasm (EPLIN), a putative metastasis suppressor, during epithelial-mesenchymal transition. J Biol Chem 288: 1469-1479.
11. Zhang S, Wang X, Osunkoya A O, Iqbal S, Wang Y, et al. (2011) EPLIN downregulation promotes epithelial-mesenchymal transition in prostate cancer cells and correlates with clinical lymph node metastasis. Oncogene 30: 4941-4952.
12. Zhau H E, Odero-Marah V, Lue H W, Nomura T, Wang R, et al. (2008) Epithelial to mesenchymal transition (EMT) in human prostate cancer: lessons learned from ARCaP model. Clin Exp Metastasis 25: 601-610.
13. Kyrgidis A, Tzellos T G (2012) Denosumab in castration-resistant prostate cancer. Lancet 379: e50; author reply e50-51.
14. Lee H J, Gallego-Ortega D, Ledger A, Schramek D, Joshi P, et al. (2013) Progesterone drives mammary secretory differentiation via RankL-mediated induction of E1f5 in luminal progenitor cells. Development 140: 1397-1401.
15. Schramek D, Leibbrandt A, Sigl V, Kenner L, Pospisilik J A, et al. (2010) Osteoclast differentiation factor RANKL controls development of progestin-driven mammary cancer. Nature 468: 98-102.
16. Chu G C Y, Zhau H E, Wang R, Rogatko A, Feng X, et al. (2013) Autocrine/paracrine RANKL-RANK signaling promotes cancer bone metastasis and establishes premetastatic niche recruiting bystander cancer cells to participate in the metastatic process. American Association for Cancer Research, 2013 Annual Meeting. Walter E. Washington Convention Center, Washington, DC. pp. #3942.
17. Ferrara N (2002) Role of vascular endothelial growth factor in physiologic and pathologic angiogenesis: therapeutic implications. Semin Oncol 29: 10-14.
18. Ferrer F A, Miller L J, Andrawis R I, Kurtzman S H, Albertsen P C, et al. (1998) Angiogenesis and prostate cancer: in vivo and in vitro expression of angiogenesis factors by prostate cancer cells. Urology 51: 161-167.
19. Forsythe J A, Jiang B H, Iyer N V, Agani F, Leung S W, et al. (1996) Activation of vascular endothelial growth factor gene transcription by hypoxia-inducible factor 1. Mol Cell Biol 16: 4604-4613.
20. Soker S, Takashima S, Miao H Q, Neufeld G, Klagsbrun M (1998) Neuropilin-1 is expressed by endothelial and tumor cells as an isoform-specific receptor for vascular endothelial growth factor. Cell 92: 735-745.
21. Whitaker G B, Limberg B J, Rosenbaum J S (2001) Vascular endothelial growth factor receptor-2 and neuropilin-1 form a receptor complex that is responsible for the differential signaling potency of VEGF(165) and VEGF(121). J Biol Chem 276: 25520-25531.
22. Talagas M, Uguen A, Garlantezec R, Fournier G, Doucet L, et al. (2013) VEGFR1 and NRP1 endothelial expressions predict distant relapse after radical prostatectomy in clinically localized prostate cancer. Anticancer Res 33: 2065-2075.
23. Zhang S, Zhau H E, Osunkoya A O, Iqbal S, Yang X, et al. (2010) Vascular endothelial growth factor regulates myeloid cell leukemia-1 expression through neuropilin-1-dependent activation of c-MET signaling in human prostate cancer cells. Mol Cancer 9: 9.
24. Hatcher D, Daniels G, Osman I, Lee P (2009) Molecular mechanisms involving prostate cancer racial disparity. Am J Transl Res 1: 235-248.
25. Thatai L C, Banerjee M, Lai Z, Vaishampayan U (2004) Racial disparity in clinical course and outcome of metastatic androgen-independent prostate cancer. Urology 64: 738-743.
26. Kwabi-Addo B, Chung W, Shen L, Ittmann M, Wheeler T, et al. (2007) Age-related DNA methylation changes in normal human prostate tissues. Clin Cancer Res 13: 3796-3802.
27. Zhau H E, Zhao L S, Chen B Q, Kojima M (1997) Interracial comparative study of prostate cancer in the United States, China, and Japan. J Cell Biochem Suppl 28-29: 182-185.
28. Zhau H E, Zhao L S, Chung L W, Chen B Q, Troncoso P, et al. (1995) Comparative studies of prostate cancers among United States, Chinese, and Japanese patients: characterization of histopathology, tumor angiogenesis, neuroendocrine factors, and p53 protein accumulations. Urol Oncol 1:51-63.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. (canceled)

2. (canceled)

3. (canceled)

4. A method of selecting a treatment for and optionally treating a cancer subject who is identified as Caucasian-American, comprising:
providing a biological sample comprising a tumor cell from the subject;
assaying the biological sample for RANKL expression level and/or NRP-1 expression level;
comparing the RANKL expression level to a RANKL reference value and/or comparing the NRP-1 expression level to a NRP-1 reference value; and
selecting a first therapy if the subject's RANKL expression level is lower than the RANKL reference value and/or the subject's NRP-1 expression level is lower than the NRP-1 reference value based on the knowledge that subjects have a high likelihood of survival if their RANKL expression level is lower than the RANKL reference value and/or NRP-1 expression level is lower than the NRP-1 reference value, or
selecting a second therapy if the subject's RANKL expression level is higher than the RANKL reference value and/or the subject's NRP-1 expression level is higher than the NRP-1 reference value based on the knowledge that subjects have a low likelihood of survival if their RANKL expression level is higher than the RANKL reference value and/or NRP-1 expression level is higher than the NRP-1 reference value.

5. The method of claim 4, further comprising administering the selected therapy.

6. The method of claim 4, wherein assaying the biological sample comprises using multispectral spectral imaging analysis.

7. The method of claim 4, wherein assaying the biological sample comprises using multiplexed quantum dot labeling imaging analysis (mQDL).

8. (canceled)

9. (canceled)

10. A method of selecting a treatment for and optionally treating a cancer subject who is identified as African-American, comprising:
identifying the subject's Gleason score;
providing a biological sample comprising a tumor cell from the subject;
assaying the biological sample for nuclear p-c-Met expression level;
comparing the nuclear p-c-Met expression level to a nuclear p-c-Met reference value; and
selecting a first therapy if the subject's Gleason score is less than 8 and the nuclear p-c-Met expression level is lower than the nuclear p-c-Met reference value based on the knowledge that subjects have a high likelihood of survival if their Gleason score is less than 8 and nuclear p-c-Met expression level is lower than the nuclear p-c-Met reference value, or
selecting a second therapy if the subject's Gleason score is >8 and the nuclear p-c-Met expression level is higher than the nuclear p-c-Met reference value based on the knowledge that subjects have a low likelihood of survival if their Gleason score is >8 and the nuclear p-c-Met expression level is higher than the nuclear p-c-Met reference value.

11. The method of claim 10, further comprising administering the selected therapy.

12. The method of claim 10, wherein assaying the biological sample comprises using multispectral spectral imaging analysis.

13. The method of claim 10, wherein assaying the biological sample comprises using multiplexed quantum dot labeling imaging analysis (mQDL).

14. The method of claim 10, wherein the first therapy is selected from the group consisting of using proactive surveillance network, dietary and life-style interventions, cholesterol lowering drug, and hormonal therapy, and the second therapy is selected from the group consisting of surgery, radiation therapy, cytotoxic chemotherapy, platinum-comprising chemotherapies, immunotherapy, bone targeted therapy, androgen receptor inhibitor, radiopharmaceutical, signal transduction inhibitor and combinations thereof.

15. (canceled)

16. (canceled)

17. (canceled)

18. (canceled)

19. (canceled)

20. (canceled)

21. A method of selecting a treatment for and optionally treating a cancer subject who is identified as Chinese, comprising:
providing a biological sample comprising a tumor cell from the subject;
assaying the biological sample for NRP-1 expression level, p-NF-κB p65 expression level, and/or VEGF expression level;
comparing the NRP-1 expression level to NRP-1 reference value, p-NF-κB p65 expression level to NF-κB p65 reference value, and/or VEGF expression level to VEGF reference value; and
selecting a first therapy if the subject's NRP-1 expression level is lower than the NRP-1 reference value, p-NF-κB p65 expression level is lower than the p-NF-κB p65 reference value, and/or VEGF expression level is lower than the VEGF reference value based on the knowledge that subjects have a high likelihood of survival if their NRP-1 expression level is lower than the NRP-1 reference value, p-NF-κB p65 expression level is lower than the p-NF-κB p65 reference value, and/or VEGF expression level is lower than the VEGF reference value, or
selecting a second therapy if the subject's NRP-1 expression level is higher than the NRP-1 reference value, p-NF-κB p65 expression level is higher than the p-NF-κB p65 reference value, and/or VEGF expression level is higher than the VEGF reference value based on the knowledge that subjects have a low likelihood of survival if their RP-1 expression level is higher than the NRP-1 reference value, p-NF-κB p65 expression level is higher than the p-NF-κB p65 reference value, and/or VEGF expression level is higher than the VEGF reference value.

22. The method of claim 21, further comprising administering the selected therapy.

23. The method of claim 21, wherein assaying the biological sample comprises using multispectral spectral imaging analysis.

24. The method of claim 21, wherein assaying the biological sample comprises using multiplexed quantum dot labeling imaging analysis (mQDL).

25. The method of claim 21, wherein the first therapy is selected from the group consisting of using proactive surveillance network, dietary and life-style interventions, cholesterol lowering drug and hormonal therapy, and the second therapy is selected from the group consisting of surgery, radiation therapy, cytotoxic chemotherapy, platinum-comprising chemotherapies, immunotherapy, bone targeted therapy, androgen receptor inhibitor, radiopharmaceutical, signal transduction inhibitor and combinations thereof.

26. (canceled)

27. A method of prognosticating cancer in a subject, optionally selecting a treatment for the subject, and optionally administering the treatment to the subject, comprising:
providing a biological sample comprising a cancer cell, a cancer-associated-stromal cell, or a morphologically normal gland cell from the subject;
wherein the biological sample comprises a cancer cell, the method comprises:
assaying the biological sample for p-c-Met expression level, RANKL expression level, and/or NRP-1 expression level;
comparing the p-c-Met expression level to a p-c-Met reference value, RANKL expression level to a RANKL reference value, and/or NRP-1 expression level to a NRP-1 reference value; and
identifying the subject as having castration resistant prostate cancer if the p-c-Met expression level is higher than the p-c-Met reference value, the RANKL expression level is higher than the RANKL reference value, and/or the NRP-1 expression level is higher than the NRP-1 reference value;
wherein the biological sample comprise the cancer-associated-stromal cell, the method comprises:
assaying the biological sample for p-c-Met expression level, RANKL expression level, and/or NRP-1 expression level;
comparing the p-c-Met expression level to a p-c-Met reference value, RANKL expression level to a RANKL reference value, and/or NRP-1 expression level to a NRP-1 reference value; and
identifying the subject as unlikely to have castration resistant prostate cancer if the p-c-Met expression level is lower than the p-c-Met reference value, the RANKL expression level is lower than the RANKL reference value, and/or the NRP-1 expression level is lower than the NRP-1 reference value, or
identifying the subject as likely having castration resistant prostate cancer if the p-c-Met expression level is higher than the p-c-Met reference value, the RANKL expression level is higher than the RANKL reference value, and/or the NRP-1 expression level is higher than the NRP-1 reference value;
wherein the biological sample comprise the morphologically normal gland cell the method comprises;
assaying the biological sample for NRP-1 expression level;
comparing the NRP-1 expression level to a NRP-1 reference value; and
identifying the subject as unlikely to have castration resistant prostate cancer if the NRP-1 expression level is lower than the NRP-1 reference value, or
identifying the subject as likely having castration resistant prostate cancer if the NRP-1 expression level is higher than the NRP-1 reference value.

28. The method of claim 27, wherein assaying the biological sample comprises using multispectral spectral imaging analysis.

29. The method of claim 27, wherein assaying the biological sample comprises using multiplexed quantum dot labeling imaging analysis (mQDL).

30. The method of claim 27, wherein the biological sample comprises a cancer cell, the method comprises selecting a treatment for the subject, comprising:
selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects are unlikely to have castration resistant prostate cancer if their p-c-Met expression level is lower than the p-c-Met reference value, RANKL expression level is lower than the RANKL reference value, and/or NRP-1 expression level is lower than the NRP-1 reference value, or
selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects likely have castration resistant prostate cancer if their p-c-Met expression level is higher than the p-c-Met reference value, RANKL expression level is higher than the RANKL reference value, and/or NRP-1 expression level is higher than the NRP-1 reference value.

31. The method of claim 30, wherein the method comprises administering the selected therapy.

32. A method of prognosticating cancer in a subject, optionally selecting a treatment for the subject, and optionally administering the treatment to the subject, comprising:
providing a biological sample comprising a cancer cell or a cancer-associated stromal cell from the subject;
wherein the biological sample comprises a cancer cell, the method comprises:
assaying the biological sample for p-c-Met expression level, RANKL expression level, and/or NRP-1 expression level;
comparing the p-c-Met expression level to a p-c-Met reference value, RANKL expression level to a RANKL reference value, and/or NRP-1 expression level to a NRP-1 reference value; and
identifying the subject as having a high likelihood of survival if the p-c-Met expression level is lower than the p-c-Met reference value, the RANKL expression level is lower than the RANKL reference value, and/or the NRP-1 expression level is lower than the NRP-1 reference value, or
identifying the subject as having low likelihood of survival if the p-c-Met expression level is higher than the p-c-Met reference value, the RANKL expression level is higher than the RANKL reference value, and/or the NRP-1 expression level is higher than the NRP-1 reference value;
wherein the biological sample comprises the cancer-associated stromal cell, the method comprises:
assaying the biological sample for p-c-Met expression level, RANKL expression level, and/or NRP-1 expression level;
comparing the p-c-Met expression level to a p-c-Met reference value, RANKL expression level to a RANKL reference value, and/or NRP-1 expression level to a NRP-1 reference value; and
identifying the subject as having a high likelihood of survival if the p-c-Met expression level is lower than the p-c-Met reference value, the RANKL expression level is lower than the RANKL reference value, and/or the NRP-1 expression level is lower than the NRP-1 reference value, or
identifying the subject as having a low likelihood of survival if the p-c-Met expression level is higher than the p-c-Met reference value, the RANKL expression level is higher than the RANKL reference value, and/or the NRP-1 expression level is higher than the NRP-1 reference value.

33. The method of claim 32, wherein assaying the biological sample comprises using multispectral spectral imaging analysis.

34. The method of claim 32, wherein assaying the biological sample comprises using multiplexed quantum dot labeling imaging analysis (mQDL).

35. The method of claim 32, wherein the biological sample comprises a cancer cell, the method comprises selecting a treatment for the subject, comprising:
selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects have a high likelihood of survival if their p-c-Met expression level is lower than the p-c-Met reference value, RANKL expression level is lower than the RANKL reference value, and/or NRP-1 expression level is lower than the NRP-1 reference value, or
selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects have a low likelihood of survival if their p-c-Met expression level is higher than the p-c-Met reference value, RANKL expression level is higher than the RANKL reference value, and/or NRP-1 expression level is higher than the NRP-1 reference value.

36. The method of claim 35, wherein the method comprises administering the selected therapy.

37. (canceled)

38. (canceled)

39. (canceled)

40. The method of claim 32, wherein the biological sample comprises the cancer-associated stromal cell, the method comprises selecting a treatment for the subject, comprising:
selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects have a high likelihood of survival if their p-c-Met expression level is lower than the p-c-Met reference value, RANKL expression level is lower than the RANKL reference value, and/or NRP-1 expression level is lower than the NRP-1 reference value, or
selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects have a low likelihood of survival if their p-c-Met expression level is higher than the p-c-Met reference value, RANKL expression level is higher than the RANKL reference value, and/or NRP-1 expression level is higher than the NRP-1 reference value.

41. The method of claim 41, wherein the method comprises administering the selected therapy.

42. (canceled)

43. (canceled)

44. (canceled)

45. The method of claim 27, wherein the biological sample comprise the cancer-associated-stromal cell, the method comprises selecting the treatment for the subject, comprising:
selecting a first therapy if the subject's p-c-Met expression level is lower than the p-c-Met reference value, RANKL expression level is lower than the RANKL reference value, and/or NRP-1 expression level is lower than the NRP-1 reference value based on the knowledge that subjects are unlikely to have castration resistant prostate cancer if their p-c-Met expression level is lower than the p-c-Met reference value, RANKL expression level is lower than the RANKL reference value, and/or NRP-1 expression level is lower than the NRP-1 reference value, or
selecting a second therapy if the subject's p-c-Met expression level is higher than the p-c-Met reference value, RANKL expression level is higher than the RANKL reference value, and/or NRP-1 expression level is higher than the NRP-1 reference value based on the knowledge that subjects likely have castration resistant prostate cancer if their p-c-Met expression level is higher than the p-c-Met reference value, the RANKL expression level is higher than the RANKL reference value, and/or the NRP-1 expression level is higher than the NRP-1 reference value.

46. The method of claim 45, wherein the method comprises administering the selected treatment.

47. A method of prognosticating cancer in a subject, optionally selecting a treatment for the subject, and optionally administering the treatment to the subject, comprising:
providing a biological sample comprising a non-cancer-associated stromal cell from the subject;
assaying the biological sample for p-c-Met expression level, and/or RANK expression level;
comparing the p-c-Met expression level to a p-c-Met reference value, RANK expression level to a RANK reference value; and
identifying the subject as having a high likelihood of survival if the p-c-Met expression level is lower than the p-c-Met reference value,
identifying the subject as having a low likelihood of survival or having castration resistant prostate cancer if the p-c-Met expression level is higher than the p-c-Met reference value, or
identifying the subject as unlikely having metastasis if the RANK expression level is lower than the RANK reference value, or
identifying the subject as likely having metastasis if the RANK expression level is higher than the RANK reference value.

48. The method of claim 47, wherein assaying the biological sample comprises using multispectral spectral imaging analysis.

49. The method of claim 47, wherein assaying the biological sample comprises using multiplexed quantum dot labeling imaging analysis (mQDL).

50. The method of claim 47, wherein the method comprises selecting the treatment, comprising:
selecting a first therapy if the subject's p-c-Met expression level is lower than the p-c-Met reference value based on the knowledge that subjects have a high likelihood of survival if their p-c-Met expression level is higher than the p-c-Met reference value, or
selecting a second therapy if the subject's p-c-Met expression level is higher than the p-c-Met reference value based on the knowledge that subjects have a low likelihood of survival if their p-c-Met expression level is higher than the p-c-Met reference value.

51. The method of claim 50, wherein the method comprises administering the selected therapy.

52. The method of claim 47, wherein the method comprises selecting the treatment, comprising:
selecting a first therapy if the subject's RANK expression level is lower than the RANK reference value based on the knowledge that subjects are unlikely to have metastasis if their RANK expression level is lower than the RANK reference value, or
selecting a second therapy if the subject's RANK expression level is higher than the RANK reference value based on the knowledge that subjects likely have metastasis if their RANK expression level is higher than the RANK reference value.

53. The method of claim 52, wherein the method comprises administering the selected therapy.

54. (canceled)

55. (canceled)

56. (canceled)

57. The method of claim 27, wherein the biological sample comprise the morphologically normal gland cell, the method comprises selecting a treatment for the subject, comprising:
selecting a first therapy if the subject's NRP-1 expression level is lower than the NRP-1 reference value based on the knowledge that subjects are unlikely to have castration resistant prostate cancer if their the NRP-1 expression level is lower than the NRP-1 reference value, or
selecting a second therapy if the subject's NRP-1 expression level is higher than the NRP-1 reference value based on the knowledge that subjects are likely to have castration resistant prostate cancer if their the NRP-1 expression level is higher than the NRP-1 reference value.

58. The method of claim 57, wherein the method comprises administering the selected treatment.

59. A system for prognosticating cancer, comprising:
a biological sample obtained from a subject who desires a prognosis regarding a cancer; and
one or more assays to determine the level of a biomarker selected from the group consisting of RANKL, NRP-1, p-c-Met, p-NF-κB p65, VEGF, RANK and combinations thereof, or
a sample analyzer configured to produce a signal for a biomarker selected from the group consisting of RANKL, NRP-1, p-c-Met, p-NF-κB p65, VEGF, RANK and combinations thereof in a biological sample of the subject; and
a computer sub-system programmed to calculate, based on the biomarker whether the signal is higher or lower than a reference value.

60. (canceled)

61. A kit for prognosticating a cancer and/or selecting a treatment for a subject in need thereof, comprising:
one or more probes comprising a combination of detectably labeled probes for the detection of RANKL, NRP-1, p-c-Met, p-NF-κB p65, VEGF, and/or RANK; and
a computer program product embodied in a non-transitory computer readable medium that, when executing on a computer, performs steps comprising:
detecting the RANKL, NRP-1, p-c-Met, p-NF-κB p65, VEGF, and/or RANK level in a biological sample from the subject; and
comparing the RANKL, NRP-1, p-c-Met, p-NF-κB p65, VEGF, and/or RANK level to a reference value.

62. (canceled)
